(19) United States
(12) Patent Application Publication
(10) Pub. No.: US 2017/0275196 A1
(43) Pub. Date: Sep. 28, 2017

(54) POROUS AND NON-POROUS BODIES

(71) Applicant: THE UNIVERSITY OF NOTTINGHAM, Nottingham, Nottinghamshire (GB)

(72) Inventor: Ifty Ahmed, Nottingham, Nottinghamshire (GB)

(21) Appl. No.: 15/505,391

(22) PCT Filed: Aug. 24, 2015
(86) PCT No.: PCT/GB2015/052446

§ 371 Date: Feb. 21, 2017

(30) Foreign Application Priority Data
Aug. 22, 2014 (GB) 1415005.6

(51) Int. Cl. C03B 19/10; A61K 45/06; A61L 27/10; C03C 11/00; A61L 27/54; A61L 27/56; C03C 12/00; A61K 9/16; A61L 27/38

(52) U.S. Cl. CPC C03B 19/1085; A61K 9/167; A61K 45/06; A61L 27/10; A61L 27/3834; A61L 27/54; A61L 27/56; C03C 12/00; C03C 11/007; C03C 11/002; A61L 2300/404; A61L 2430/02

(57) ABSTRACT

A method of manufacture of a powder comprising, or consisting essentially of, microspheres, the method comprising: providing a feed powder; and applying at least one spheroidisation flame to the powder. The powder may be suitable for use in medical and/or non-medical applications.

The invention relates to powders comprising porous and/or non-porous bodies, in particular porous or non-porous spherical bodies or microspheres. The invention also relates to methods of manufacture of and the use of powders comprising porous and/or non-porous bodies such as resorbable porous microspheres.

Porous bodies can be useful in a range of applications including in the delivery of biological cells, growth factors, proteins and pharmaceutically active agents. By making the porous bodies from a resorbable material with controlled degradation, the resorbable bodies may be resorbed by their in situ environment over time. For instance, a bio-resorbable material may be suitable for being resorbed within a human or animal body. Consequently, a temporary medical device made from a bio-resorbable material may be left in the body to be resorbed over time. During resorption of the temporary medical device, specific and/or therapeutic agents, e.g. ions, may be released in a controlled manner.

The potential uses for resorbable and non-resorbable microspheres are many and varied. However, a reliable and reproducible method for manufacturing significant volumes of suitable porous microspheres has not yet been developed. Previous methods of manufacturing porous microspheres have generally been unsatisfactory. Typically, yields have been poor in terms of sphericity and/or porosity. The methods may also have relatively poor reliability and/or may produce microspheres lacking in uniformity. The methods may also be time consuming and/or may comprise several steps pre- and/or post-microsphere production.

A first aspect of the invention provides a powder comprising, or consisting essentially of, porous and/or non-porous microspheres.

Optionally, the microspheres may have an average particle size of at least 30 μm and/or up to 500 μm. In an embodiment, the microspheres may have an average particle size of at least 50 μm and/or an average particle size of up to 400 μm or up to 350 μm.

The microspheres may comprise a resorbable composition, e.g. an at least partially resorbable composition or a fully resorbable composition, or a non-resorbable composition. The microspheres may be biocompatible and/or bio-resorbable.

In an embodiment, the microspheres may comprise a glass, a glass-ceramic or a ceramic composition.

For instance, the microspheres may comprise a phosphate-based glass such as a calcium phosphate-based glass. The phosphate-based glass may be doped with an amount of one of more oxides, e.g. Na₂O, K₂O, MgO, CaO, SrO, CuO, Cu₂O, CoO, AgO, Ag₂O, ZnO, SiO₂, Ga₂O₃, B₂O₃, Fe₂O₃ or TiO₂.

Phosphate-based glasses may be particularly well suited to use in bone regeneration and repair. Phosphate-based glasses have been shown to be bio-compatible with bone, the main chemical constituent of which is a calcium phosphate.

The phosphate-based glass typically may comprise or consist essentially of P₂O₅, CaO and/or Na₂O. The phosphate-based glass may be doped with one or more network formers such as SiO₂ or B₂O₃ and/or one or more network modifier oxides, e.g. K₂O, Rb₂O, MgO, SrO, ZnO, AgO, CuO, Cu₂O, CoO, Ag₂O, ZnO, Fe₂O₃ or TiO₂. The phosphate-based glass may include silica and/or boron and/or germanium.

An advantage of phosphate-based glasses is that they may be totally soluble. In addition, the dissolution rate may be varied and/or controlled by increasing and/or decreasing the relative proportions of the oxide components, e.g. P₂O₅, CaO and/or Na₂O and/or the network former(s) and/or the network modifier(s).

In an embodiment, the phosphate-based glass may comprise up to or at least 16 mol % SrO.

The microspheres may comprise Bioglass®, typically a silicate-based Bioglass® such as 45S5 or 13-93. Typically, microspheres comprising a phosphate-based glass or Bioglass® may be resorbable over time.

The microspheres may comprise hydroxyapatite, a tri-calcium phosphate (α-TCP), tri-calcium phosphate (β-TCP), a borosilicate glass, a borate glass or a glass-ceramic such as apatite wollastonite. Typically, microspheres comprising hydroxyapatite, a tri-calcium phosphate (α-TCP), β tri-calcium phosphate (β-TCP), a borosilicate glass, or apatite wollastonite may be non-resorbable.

In an embodiment, the microspheres may contain strontium. Strontium may be present in the microspheres in an amount of up to around 7 wt % or up to around 6 wt %.

The microspheres may have a surface area per unit mass of at least 0.05 m²/g. In an embodiment, the microspheres may have a surface area per unit mass of up to or at least 0.08 m²/g, up to or at least 0.12 m²/g or up to or at least 0.14 m²/g.

In an embodiment, the microspheres may be porous and the average pore diameter may be at least 10 μm and/or up to 100 μm. The average pore diameter may be up to or at least 30 μm, up to or at least 40 μm, up to or at least 50 μm, up to or at least 60 μm, up to or at least 70 μm or up to or at least 80 μm.

In an embodiment, the microspheres may have a total porosity of at least 40%, at least 50%, at least 60%, at least 70% or at least 80%.

The microspheres may comprise at least some interconnected porosity.

In an embodiment, the microspheres may comprise surface porosity. For example, the microspheres may comprise only surface porosity.

In an embodiment, the powder may comprise a mixture of a first powder and at least one further powder, wherein the first powder comprises microspheres having a first size distribution and the or each further powder comprises microspheres having a different size distribution.

The first powder and the or each further powder may be mixed together in any ratio.

In an embodiment, the microspheres of the first powder may be smaller than the microspheres of a second powder. The microspheres of the first powder may have an average particle size of up to 200 μm and/or the microspheres of the second powder may have an average particle size of more than 200 μm.

In an embodiment, the microspheres of the first powder may have an average particle size of from 50 μm and/or up to 150 μm. The microspheres of the first powder may have an average particle size of up to or at least 60 μm and/or up to or at least 140 μm.

In an embodiment, the microspheres of the second powder may have an average particle size of up to 400 μm. The microspheres of the second powder may have an average particle size of up to or at least 250 μm and/or up to or at least 350 μm.

In an embodiment, the microspheres may be coated and/or loaded with at least one active agent, e.g. a pharmaceutically active agent. For instance, the porous microspheres may be loaded with biological cells, e.g. stem cells, growth factors, proteins and/or other biological components.

In an embodiment, the microspheres may be coated at least in part. For example, the microspheres may have a coating comprising an anti-microbial composition and/or an antibacterial composition, a resorbable polymer or a non-resorbable polymer.

The microspheres may be impregnated or doped with an anti-microbial agent and/or an antibacterial agent. The anti-microbial or antibacterial agent may comprise one or more of silver, zinc and/or copper.

In an embodiment, the microspheres may be hollow.

A second aspect of the invention provides a method of manufacture of a powder comprising, or consisting essentially of, microspheres, the method comprising:
providing a feed powder; and
applying at least one spheroidisation flame to the powder.

In some embodiments, the method may comprise the steps of: mixing the feed powder with one or more blowing agents to provide a mixture; and applying at least one spheroidisation flame to the mixture.

The feed powder may comprise porous and/or non-porous particles. The feed powder particles may be resorbable or non-resorbable. The feed powder may comprise substantially spherical and/or non-spherical particles. The feed powder may have an average particle size of from 30 μm to 500 μm. In an embodiment, the feed powder may have an average particle size of at least 50 μm and/or an average particle size of up to 400 μm or up to 350 μm.

In an embodiment, the microspheres may comprise a glass, a ceramic or a glass-ceramic composition.

The microspheres may comprise a resorbable, e.g. a bio-resorbable composition, or a non-resorbable composition. The microspheres may comprise an at least partially resorbable composition or a fully resorbable composition. The microspheres may comprise a biocompatible composition.

The feed powder or the mixture may be passed through the spheroidisation flame.

The method of manufacture may comprise flame-spraying spheroidisation. In flame-spraying spheroidisation, the feed powder or the mixture may be sprayed into and/or through the spheroidisation flame. Typically, flame-spraying spheroidisation may produce a good yield of relatively uniform, highly spherical microspheres.

The sphericity and the porosity of the porous microspheres manufactured in accordance with the invention may depend on a number of factors, including the size and temperature of the spheroidisation flame and the residence time of the mixture within the spheroidisation flame. Accordingly, the size and/or temperature of the flame and/or the residence time may be controlled and/or varied in order to manufacture porous microspheres having desired properties.

The spheroidisation flame may be applied to the mixture for a predetermined period of time.

The spheroidisation flame temperature may be from 1900° C. to 3400° C., depending on the type and ratio of fuel used.

An oxygen:butane spheroidisation flame may have a temperature of around 1920° C. An oxygen:propane spheroidisation flame may have a temperature of around 2800° C. An oxygen:acetylene spheroidisation flame may have a temperature of around 3400° C.

The spheroidisation flame may be produced by an acetylene torch or a flame spray gun such as a plasma spray gun.

In an embodiment, the spheroidisation flame may be produced by an acetylene torch using an oxygen to acetylene ratio of 4:3.

The or each blowing agent may have an average particle size of at least 5 μm and/or up to 500 μm. By varying the particle size of the blowing agent(s), the size of the pores in the microspheres may be controlled. Different pore sizes may be achieved by using differently sized blowing agent particles. The size of the blowing agent particles may be varied.

The or each blowing agent may comprise a carbonate or a sulphate. For instance, suitable blowing agents may include calcium carbonate, strontium carbonate, zinc carbonate, magnesium carbonate, sodium sulphate and/or calcium sulphate. The type and/or amount of blowing agent(s) utilised can be used to control the levels of porosity and pore sizes of the porous microspheres manufactured in accordance with the invention. Accordingly, different types and/or amounts of blowing agent(s) may be selected in order to manufacture porous microspheres having desired properties.

In an embodiment, the ratio by weight of the blowing agent(s) to the feed powder particles may be from 5:1 to 1:10.

The mixture may be produced prior to applying the spheroidisation flame or at the same time as applying the spheroidisation flame. For instance, the mixture may have been formed before being supplied to a spray head configured to spray the mixture through the spheroidisation flame. Alternatively, the mixture could be formed at the spray head, e.g. by supplying the feed powder and the blowing agent(s) separately to the spray head. Alternatively, the mixture could be formed during spraying, e.g. by spraying the feed powder through a first spray head and the blowing agent(s) through one or more further spray heads such that the feed powder and the blowing agent(s) can mix together.

The method may comprise the step of coating the feed powder with the blowing agent(s).

The method may comprise the step of soaking the feed powder in a solution containing the blowing agent(s).

The solution containing the blowing agent(s) may be an aqueous solution.

The feed powder may be soaked in the solution containing the blowing agent(s) for a period of at least a few minutes (e.g. five minutes) and/or up to several hours (e.g. 6 hours or 12 hours).

Advantageously, soaking the feed powder in a solution containing the blowing agent(s) may degrade or attack the surface of the feed powder particles, thereby making the particles “sticky”. Consequently, the blowing agent(s) may stick to the surface of the feed powder particles. Hence, the interaction between the blowing agent(s) and the powder particles as the spheroidisation flame is applied to the mixture may be improved.

In an embodiment, an agent may be utilised to make the surface of the feed powder particles “sticky” for the blowing agent(s). An example of a suitable agent is water soluble cellulose or a weak acid.

Advantageously, bubbles of gas generated by the blowing agent(s) may form more pores and/or generally larger pores in the powder particles, if the blowing agent(s) were stuck to the surface of the powder particles, e.g. following soaking of the feed powder in a solution containing the blowing agent(s) or coating of the feed powder with the blowing agent(s).

Acceptable porosity characteristics may also be realised without soaking the feed powder in a solution containing the blowing agent(s) or coating the feed powder with the blowing agent(s).

In an embodiment, the method may comprise a washing step to remove residual blowing agent(s). Typically, the washing step may be carried out after the step of applying the spheroidisation flame.

Advantageously, the washing step may also help to control porosity of the microspheres. The washing step may help to increase the size of surface pores and/or may enhance interconnected porosity.

The washing step may comprise washing the microspheres in an acidic solution. The acidic solution may comprise, for example, acetic acid.

The washing step may comprise soaking the microspheres in a fluid, e.g. an acidic solution. Further control of porosity, e.g. pore size, may be achieved by varying the length of time the microspheres are left to soak in the fluid. When an acidic solution is used, further control of porosity, e.g. pore size or interconnected porosity, may be achieved by varying the concentration of the acidic solution.

A third aspect of the invention provides a method of manufacture of a powder comprising, or consisting essentially of, microspheres, the method comprising:
manufacturing a first powder according to the second aspect of the invention;
manufacturing at least one further powder according to the second aspect of the invention, wherein the at least one further powder contains particles having a different size distribution and/or a different porosity from the first powder; and
mixing the first powder and the at least one further powder together.

The first powder and the further powder(s) may be mixed together in any ratio. The resulting powder may have any desired proportion of particles with particular size distributions and/or porosities.

A fourth aspect of the invention provides a use of a powder according to the first aspect of the invention or the use of a powder manufactured according to the second aspect or the third aspect of the invention. The use may be a medical or a non-medical use. For instance, porous microspheres may be loaded with autologous stem cells and used to promote bone tissue repair and regeneration. Alternatively, porous microspheres may be used to filter one or more entities out of a solution. Alternatively, the powder may be used as a feedstock for a manufacturing process, e.g. an additive manufacturing process such as three-dimensional printing.

A fifth aspect of the invention provides a method of treatment of osteoporosis comprising:
identifying an individual having or at risk of having osteoporosis;
examining the individual to identify one or more region(s) of resorbed osteoporotic bone;
isolating autologous stem cells from the individual;
loading and/or coating a powder according to the first aspect of the invention or a powder manufactured according to the second aspect or the third aspect of the invention with the isolated autologous stem cells; and
delivering the powder loaded and/or coated with the autologous stem cells to the region(s) of resorbed osteoporotic bone.

The powder loaded and/or coated with the autologous stem cells may be delivered to the region(s) of resorbed osteoporotic bone via a minimally invasive route, a non-invasive route or a non-minimally invasive route.

The individual may have a fracture or be at risk of having a fracture due to osteoporosis.

The method may be used to treat osteoporosis, e.g. before fracture, in a human or an animal. Alternatively or additionally, the method may be used to prevent or at least reduce the likelihood of further fractures, e.g. in the spine, hip, arm, leg, wrist, ankle etc.

A sixth aspect of the invention provides a method of manufacturing a component, product or part thereof, the method comprising: supplying a feedstock comprising a powder according to the first aspect of the invention or a powder manufactured according to the second aspect or the third aspect of the invention to an additive manufacturing device; and operating the additive manufacturing device to produce the component, product or part thereof. The additive manufacturing device may comprise a three-dimensional printer.

A seventh aspect of the invention provides a computer-readable medium having computer-executable instructions adapted to cause an additive manufacturing device such as a 3D printer to produce a component, product or part thereof from a feedstock comprising a powder according to the first aspect of the invention or a powder manufactured according to the second aspect or the third aspect of the invention.

In order that the invention may be well understood, it will now be described by way of example only with reference to the accompanying drawings in which:

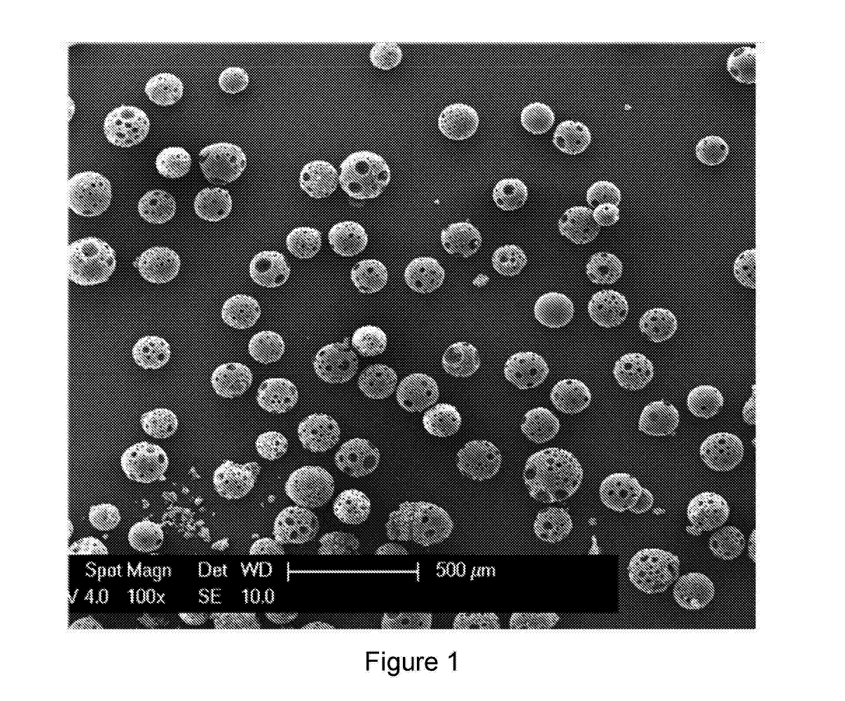

FIG. 1 is a scanning electron microscope (SEM) image of a plurality of porous resorbable microspheres according to a first example embodiment of the invention;

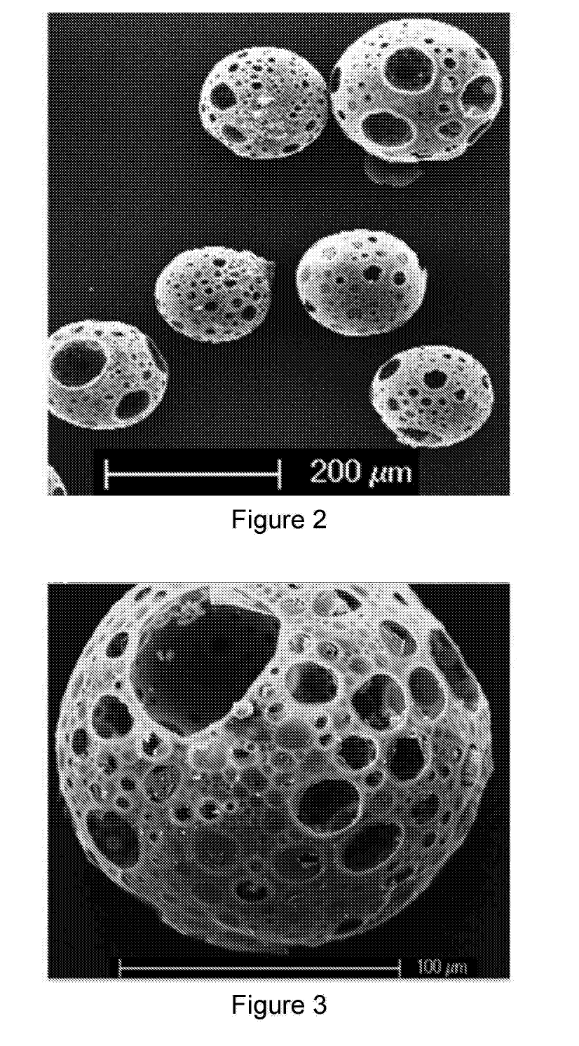

FIG. 2 is a higher magnification SEM image of some of the porous resorbable microspheres shown in FIG. 1;

FIG. 3 is an SEM image of one of the porous resorbable microspheres shown in FIG. 1;

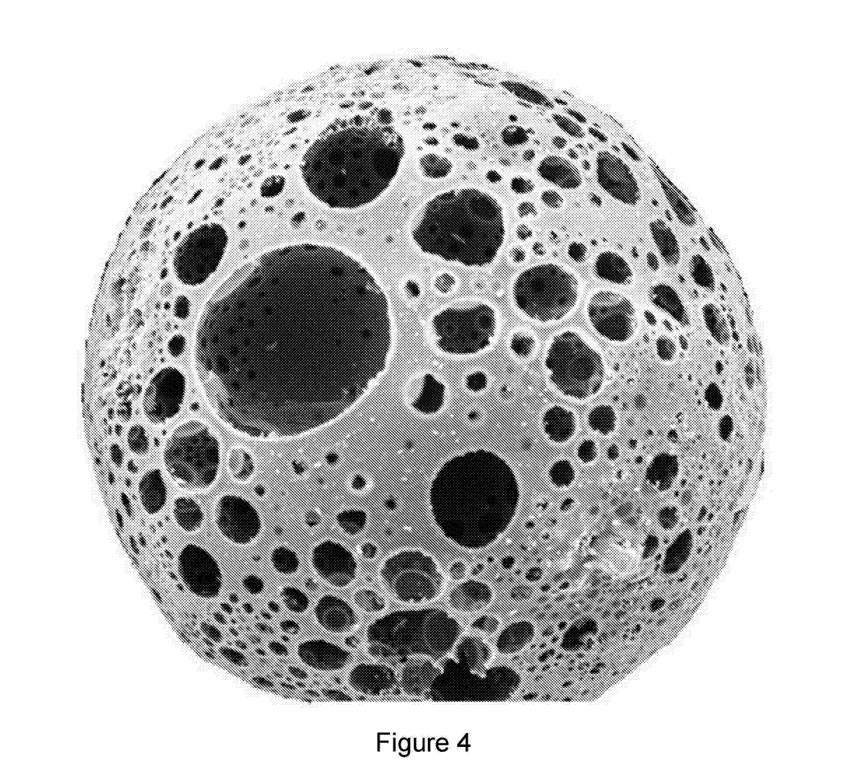

FIG. 4 is an SEM image of one of the porous resorbable microspheres shown in FIG. 1;

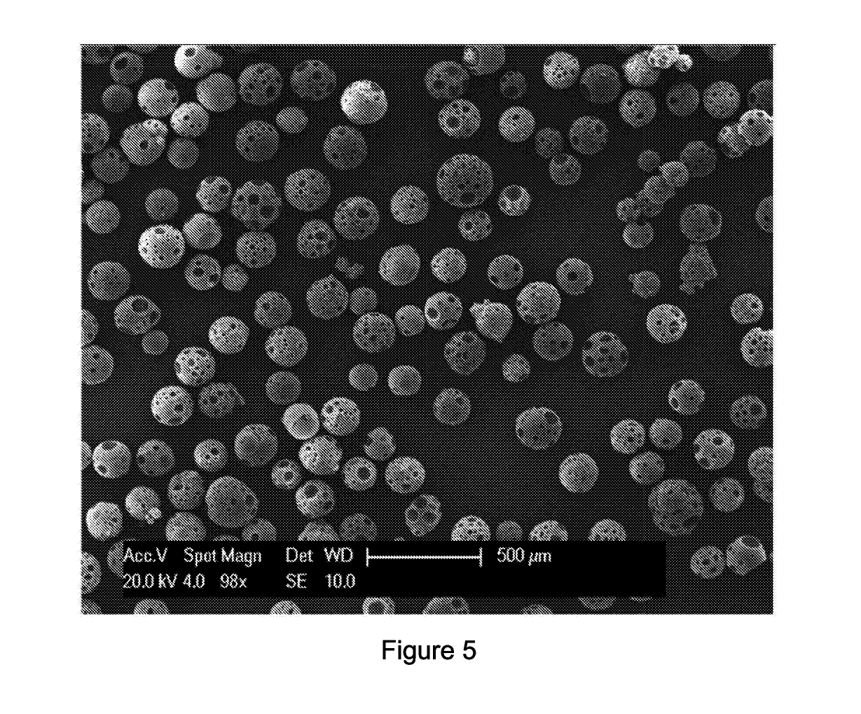

FIG. 5 is an SEM image of a plurality of porous resorbable microspheres according to a second example embodiment of the invention;

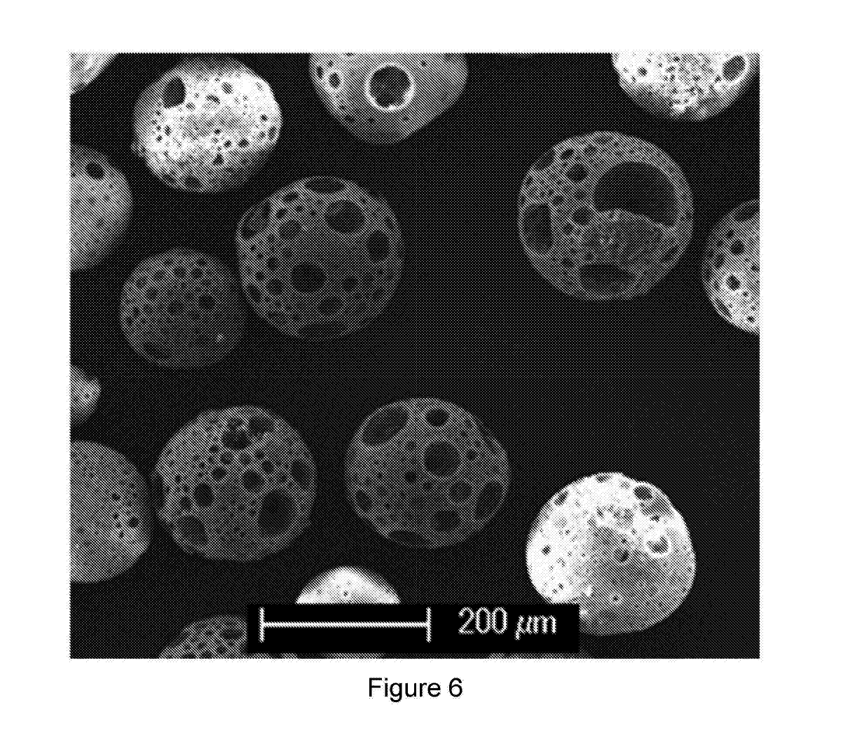

FIG. 6 is a higher magnification SEM image of some of the porous resorbable microspheres shown in FIG. 5;

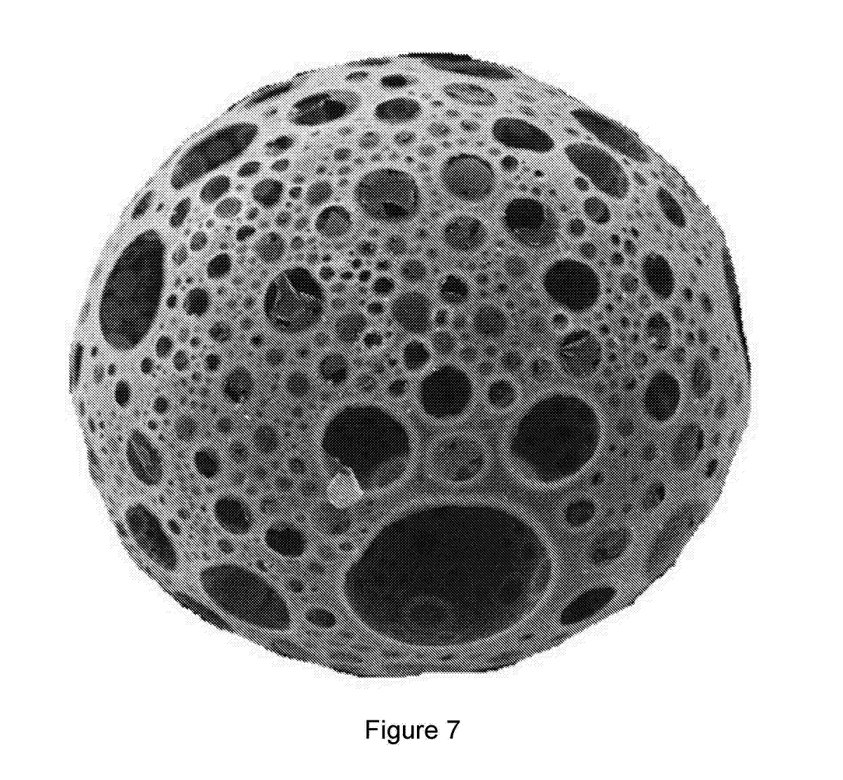

FIG. 7 is an SEM image of one of the porous resorbable microspheres shown in FIG. 5;

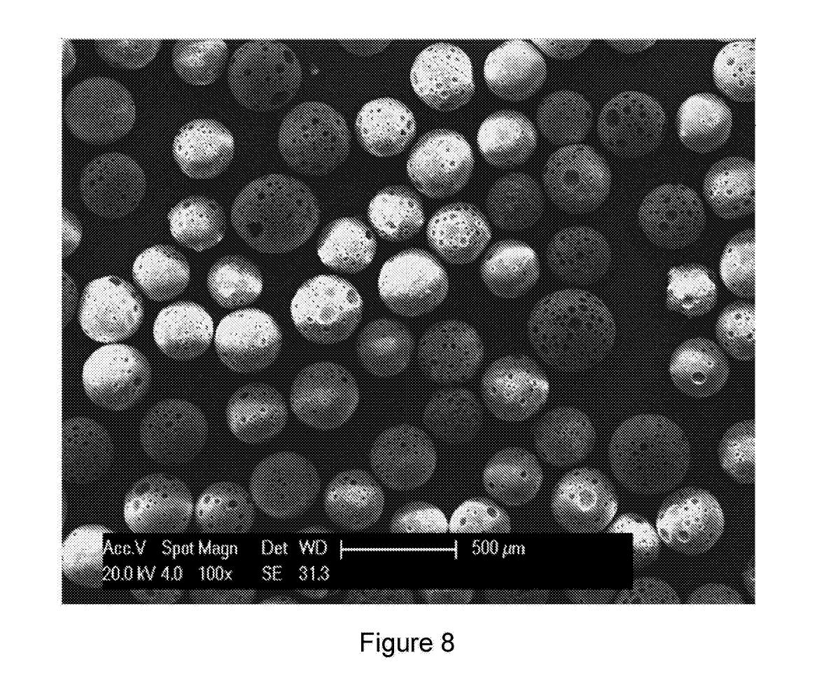

FIG. 8 is an SEM image of a plurality of porous resorbable microspheres according to a third example embodiment of the invention;

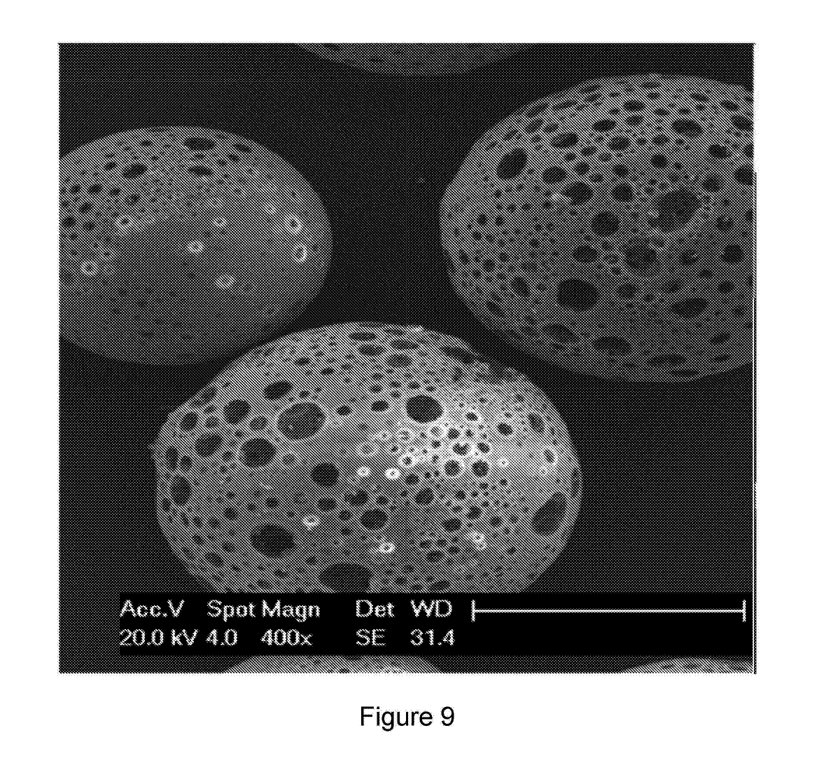

FIG. 9 is a higher magnification SEM image of some of the porous resorbable microspheres shown in FIG. 8;

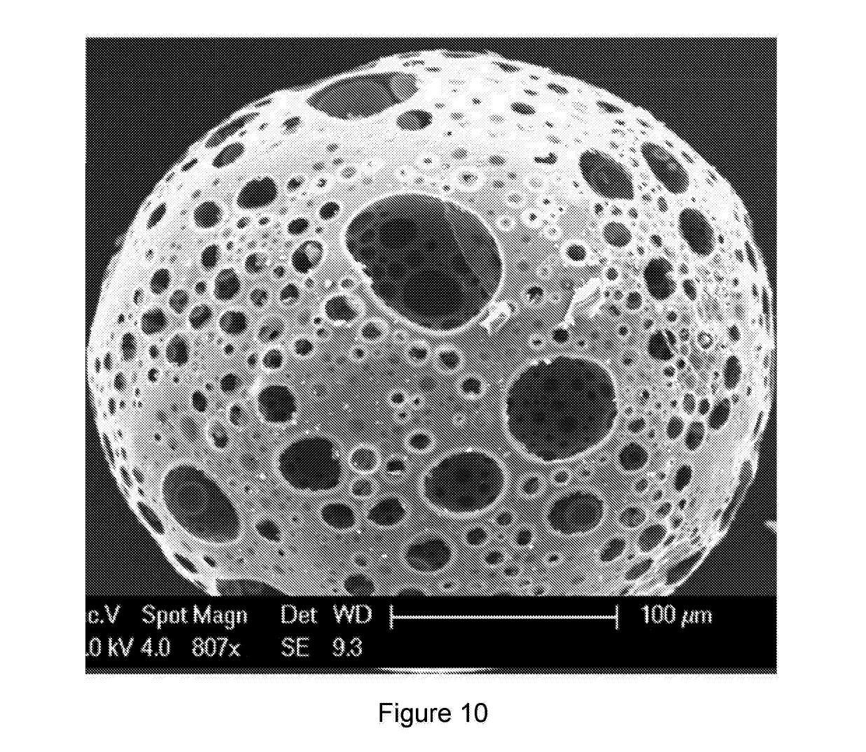

FIG. 10 is an SEM image of one of the porous resorbable microspheres shown in FIG. 8;

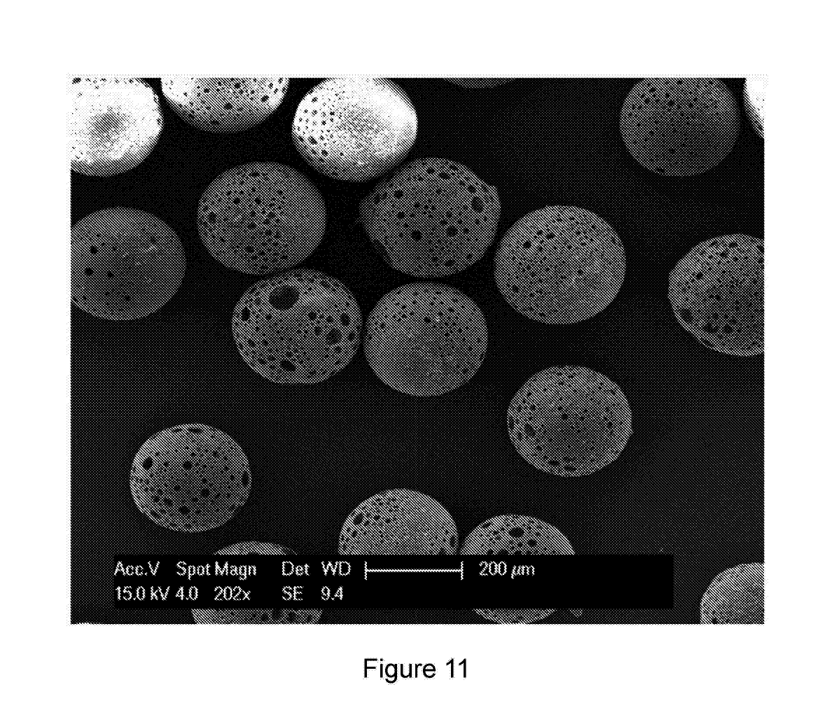

FIG. 11 is an SEM image of a plurality of porous resorbable microspheres according to a fourth example embodiment of the invention;

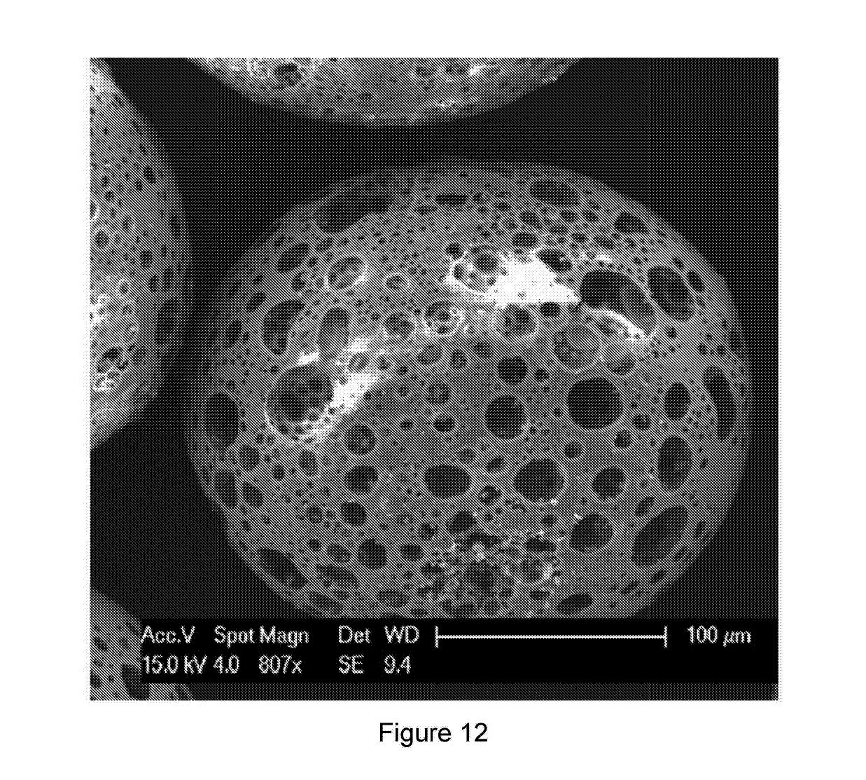

FIG. 12 is an SEM image of one of the porous resorbable microspheres shown in FIG. 11;

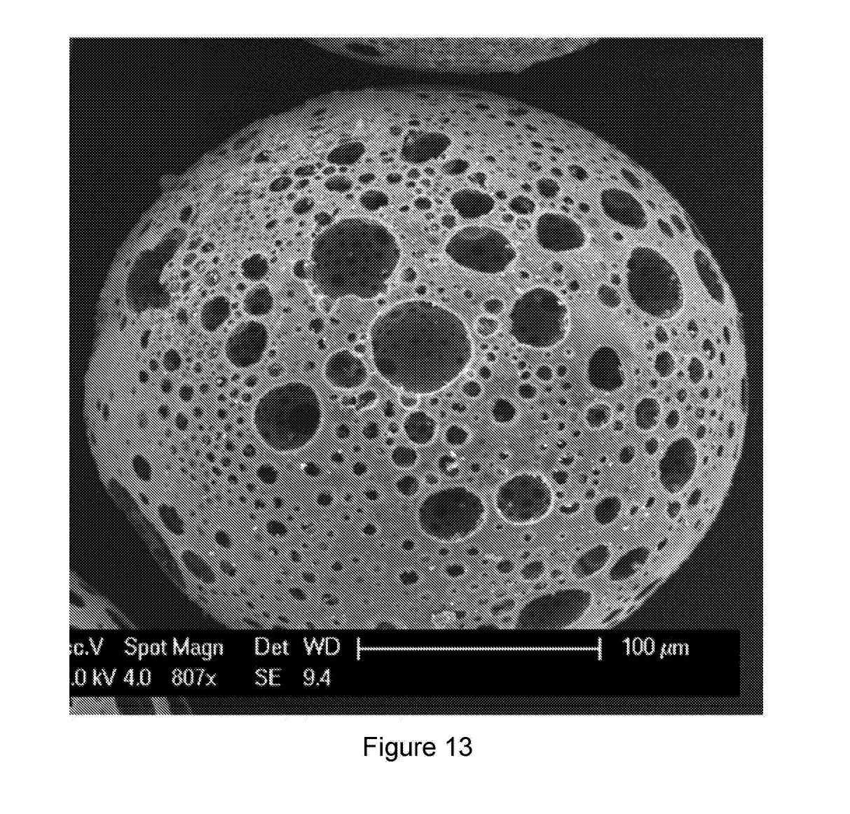

FIG. 13 is an SEM image of one of the porous resorbable microspheres shown in FIG. 11;

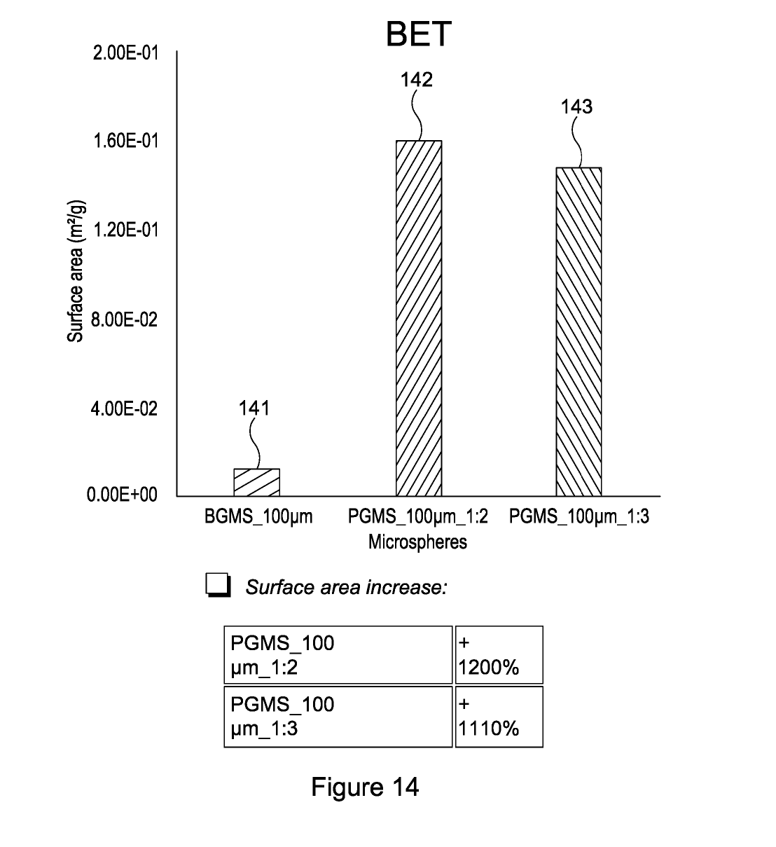

FIG. 14 is a bar chart showing the results of surface area analysis carried out on two example embodiments of porous resorbable microspheres according to the invention;

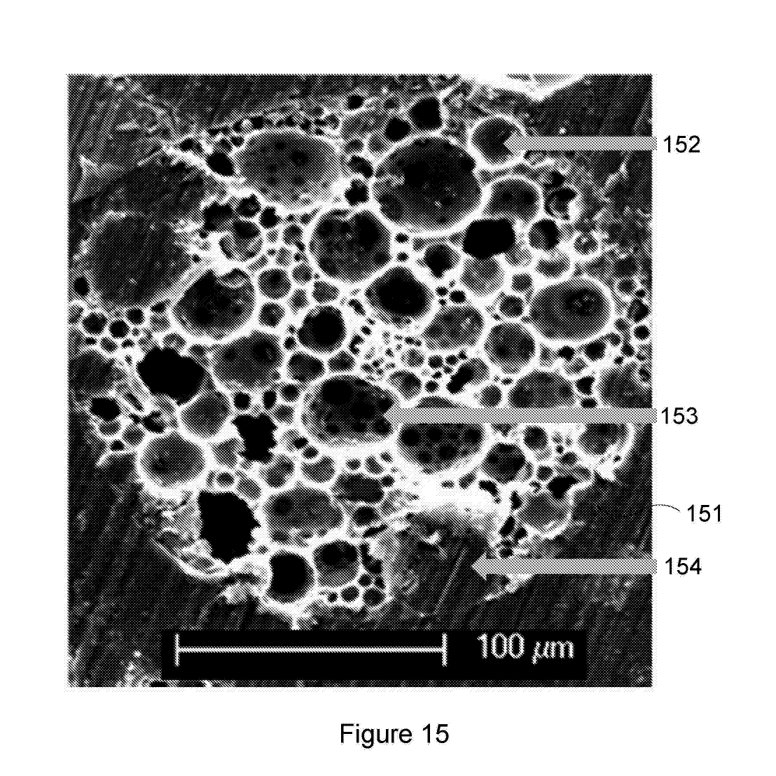

FIG. 15 is an SEM image of a cross-section of a porous resorbable microsphere according to the invention;

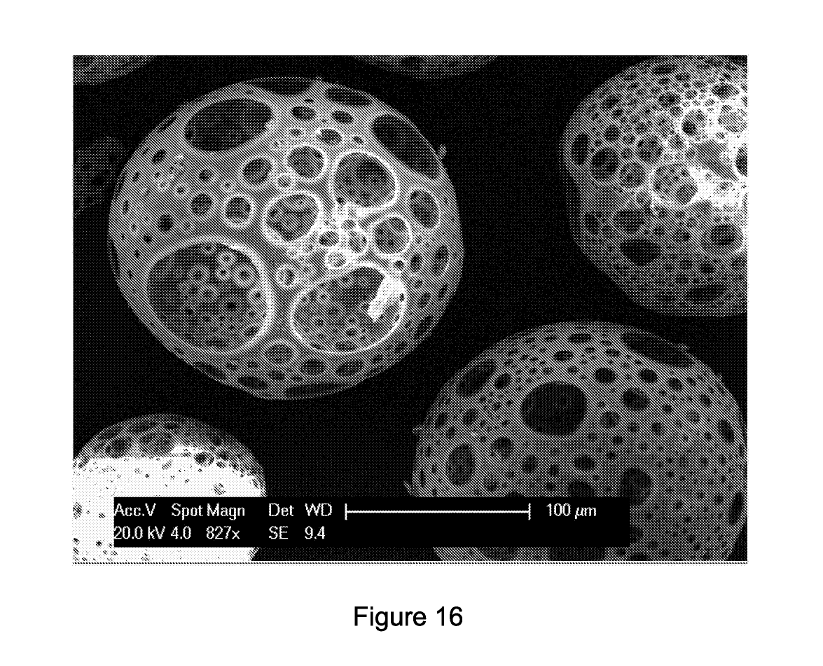

FIG. 16 is an SEM image of an example embodiment of acid washed porous microspheres according to the invention;

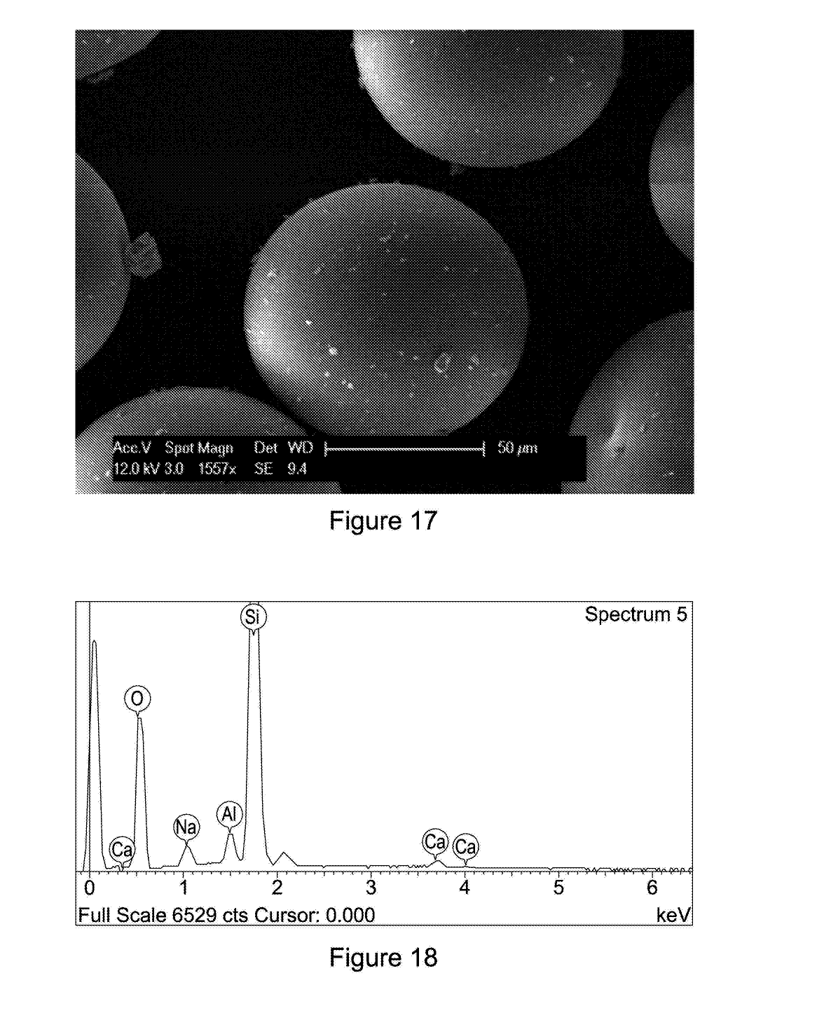

FIG. 17 is an SEM image of a plurality of non-porous borosilicate microspheres according to another example embodiment of the invention;

FIG. 18 is an energy dispersive x-ray (EDX) spectrum for the non-porous borosilicate microspheres shown in FIG. 17;

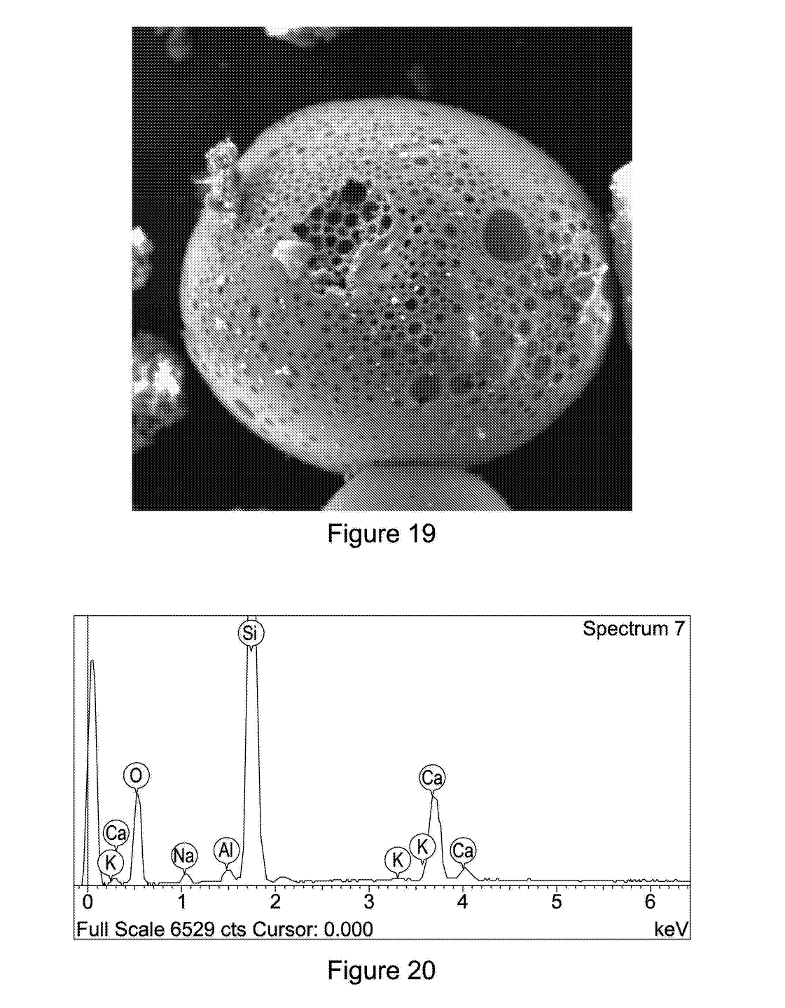

FIG. 19 is an SEM image of a porous borosilicate microsphere according to another example embodiment of the invention;

FIG. 20 is an EDX spectrum for the porous borosilicate microspheres shown in FIG. 19;

FIG. 21 is an SEM image of a plurality of non-porous Bioglass® microspheres according to another example embodiment of the invention;

FIG. 22 is an EDX spectrum for the non-porous Bioglass® microspheres shown in FIG. 21;

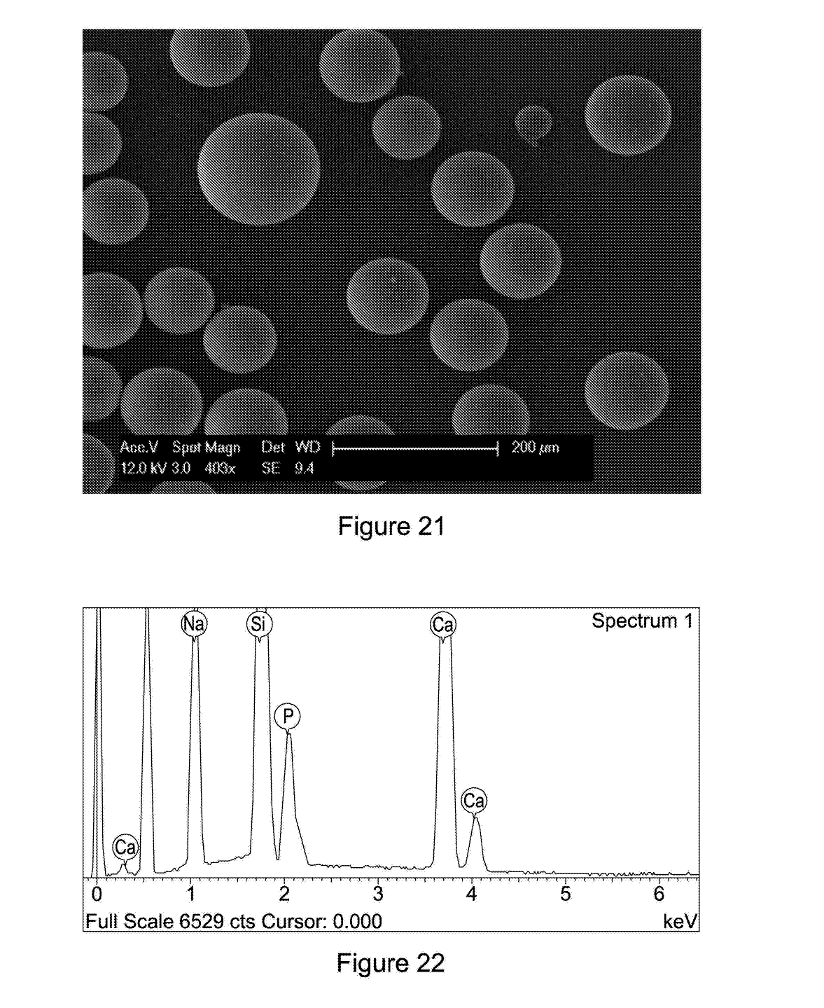

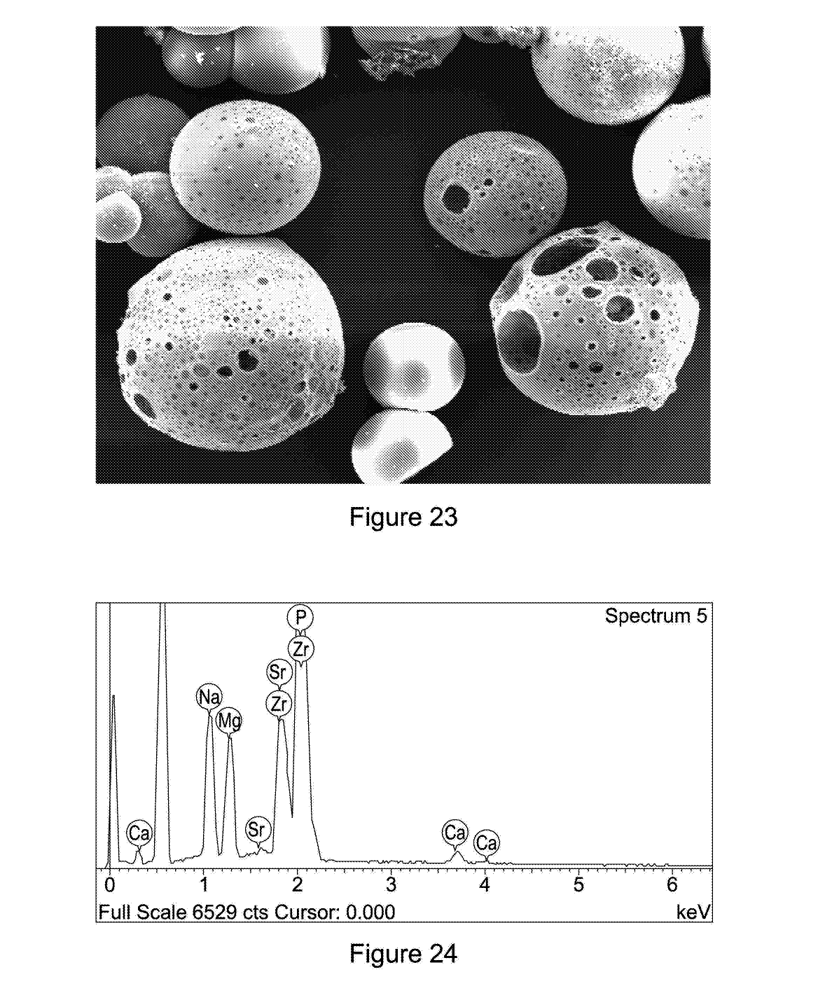

FIG. 23 is an SEM image of a plurality of porous Bioglass® microspheres according to another example embodiment of the invention;

FIG. 24 is an EDX spectrum for the porous Bioglass® microspheres shown in FIG. 23;

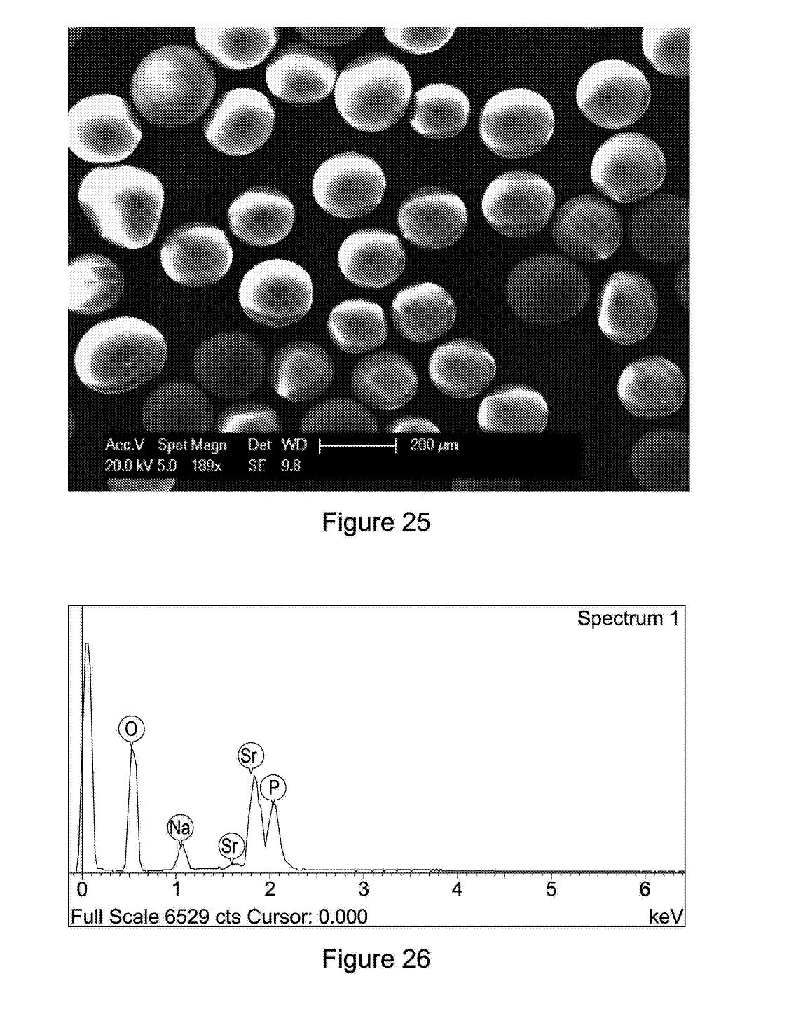

FIG. 25 is an SEM image of a plurality of non-porous borate glass microspheres according to another example embodiment of the invention;

FIG. 26 is an EDX spectrum for the non-porous borate glass microspheres shown in FIG. 25;

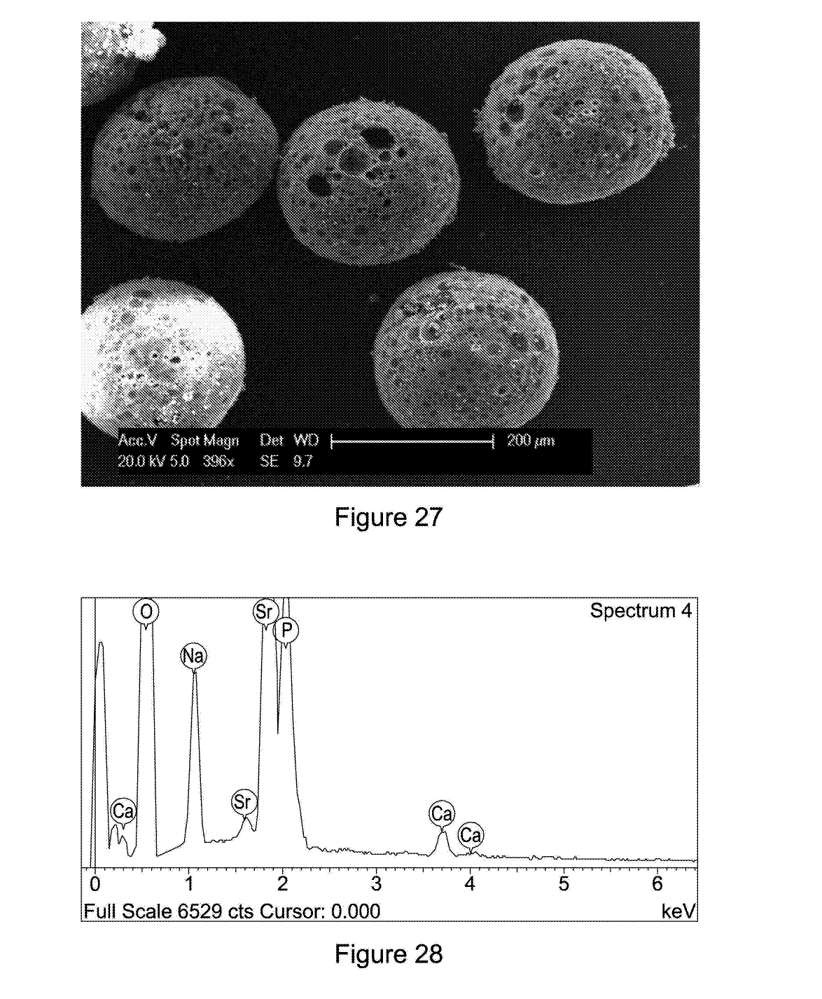

FIG. 27 is an SEM image of a plurality of porous borate glass microspheres according to another example embodiment of the invention;

FIG. 28 is an EDX spectrum for the porous borate glass microspheres shown in FIG. 27;

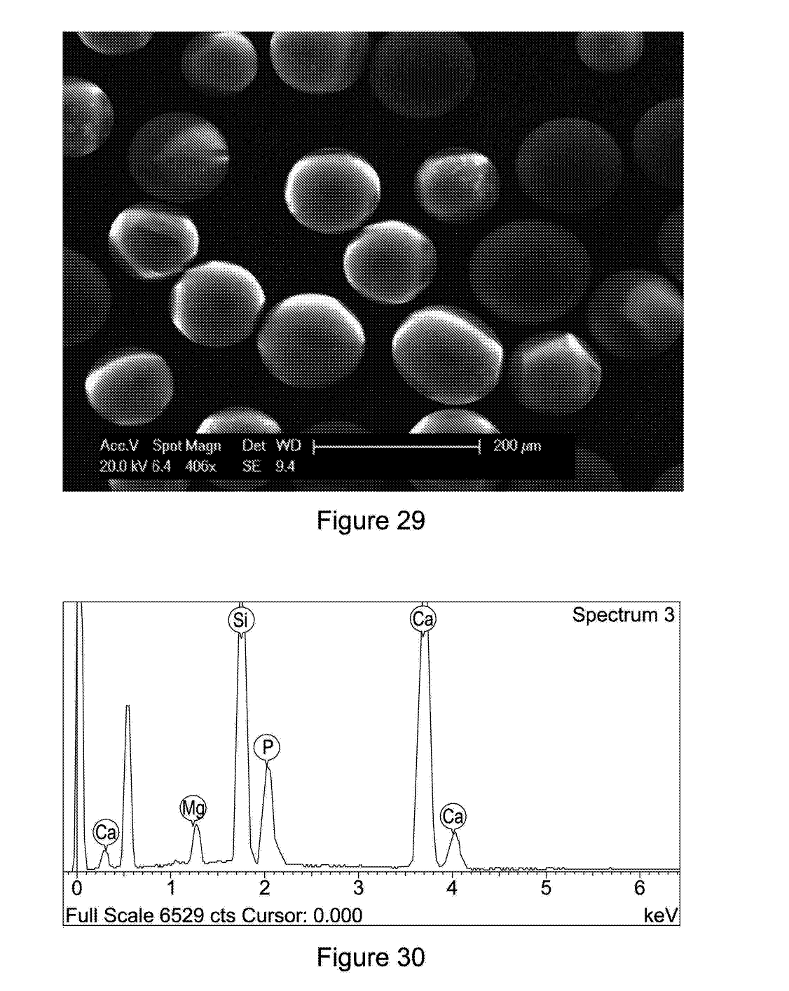

FIG. 29 is an SEM image of a plurality of non-porous apatite wollastonite microspheres according to another example embodiment of the invention;

FIG. 30 is an EDX spectrum for the non-porous apatite wollastonite microspheres shown in FIG. 29;

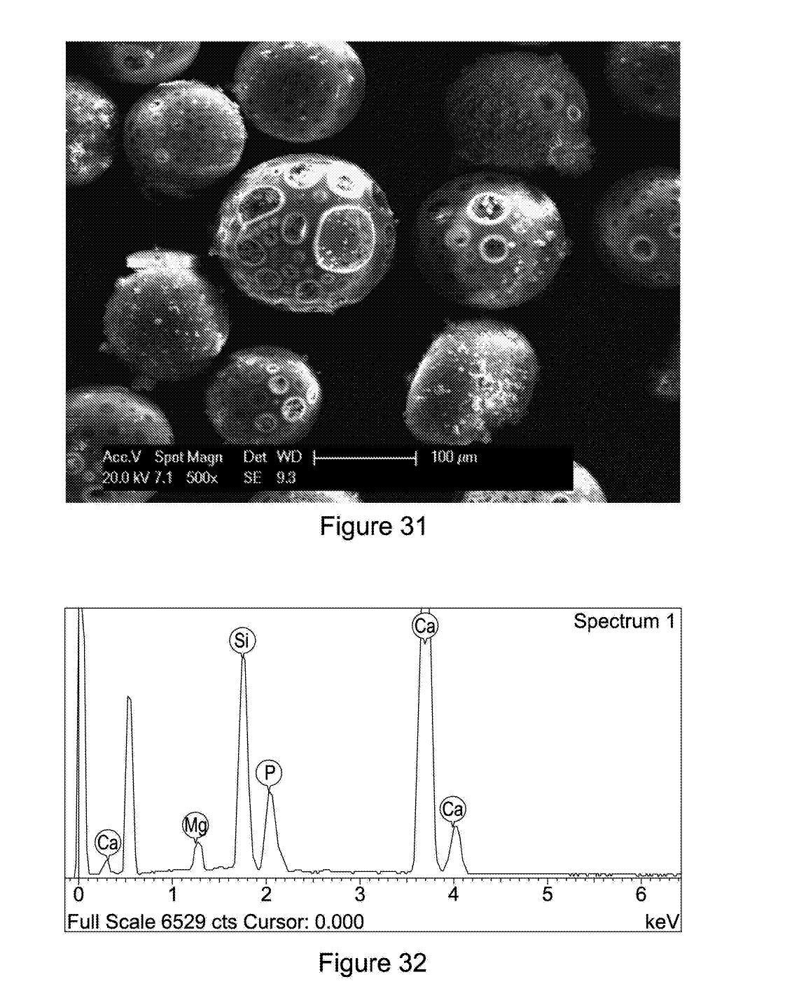

FIG. 31 is an SEM image of a plurality of porous apatite wollastonite microspheres according to another example embodiment of the invention;

FIG. 32 is an EDX spectrum for the porous apatite wollastonite microspheres shown in FIG. 31;

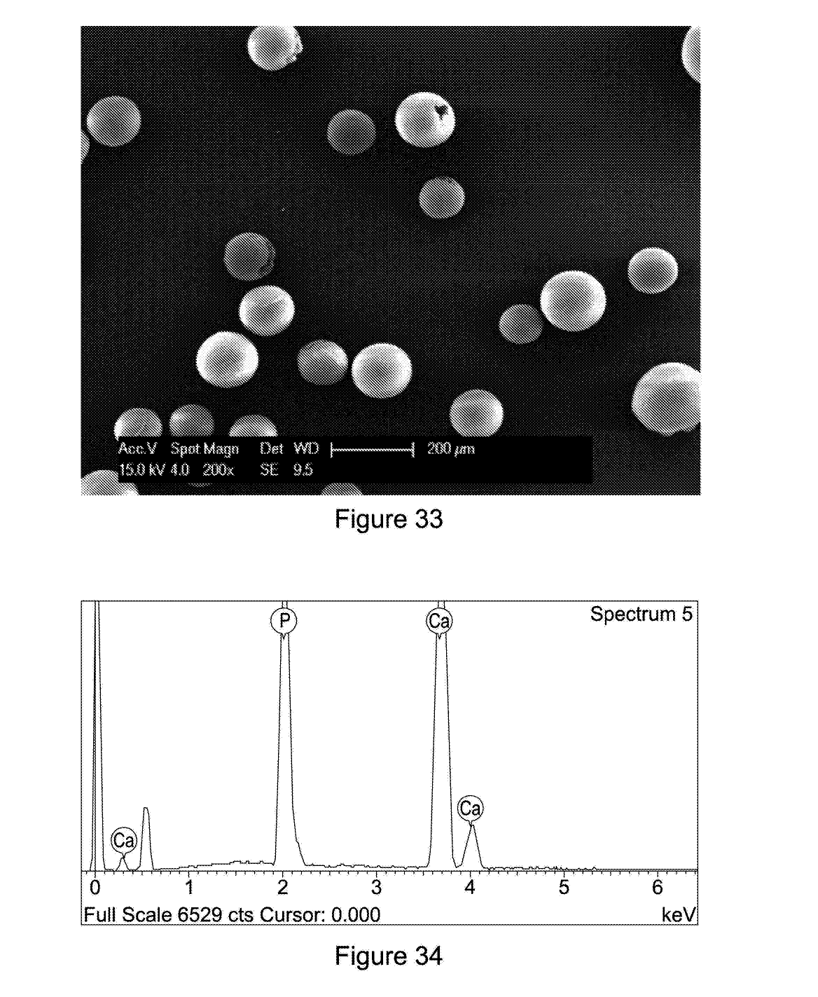

FIG. 33 is an SEM image of a plurality of non-porous hydroxyapatite microspheres according to another example embodiment of the invention;

FIG. 34 is an EDX spectrum for the non-porous hydroxyapatite microspheres shown in FIG. 33;

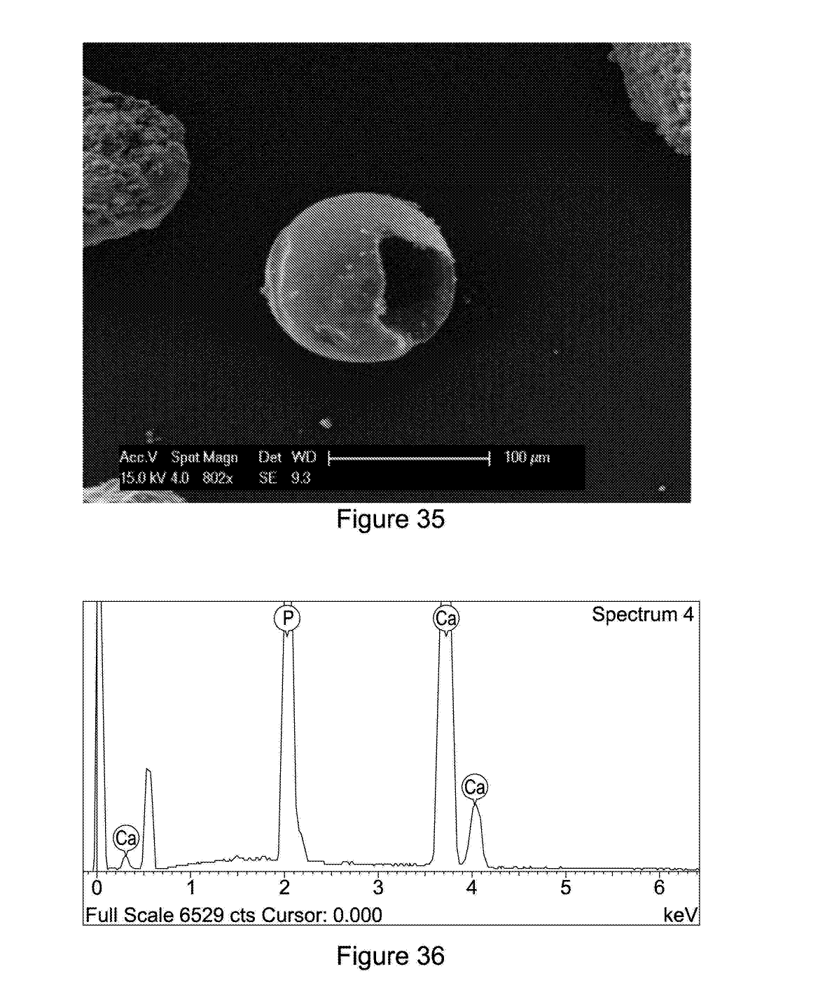

FIG. 35 is an SEM image of a hollow hydroxyapatite microsphere according to another example embodiment of the invention;

FIG. 36 is an EDX spectrum for the hollow hydroxyapatite microsphere shown in FIG. 35;

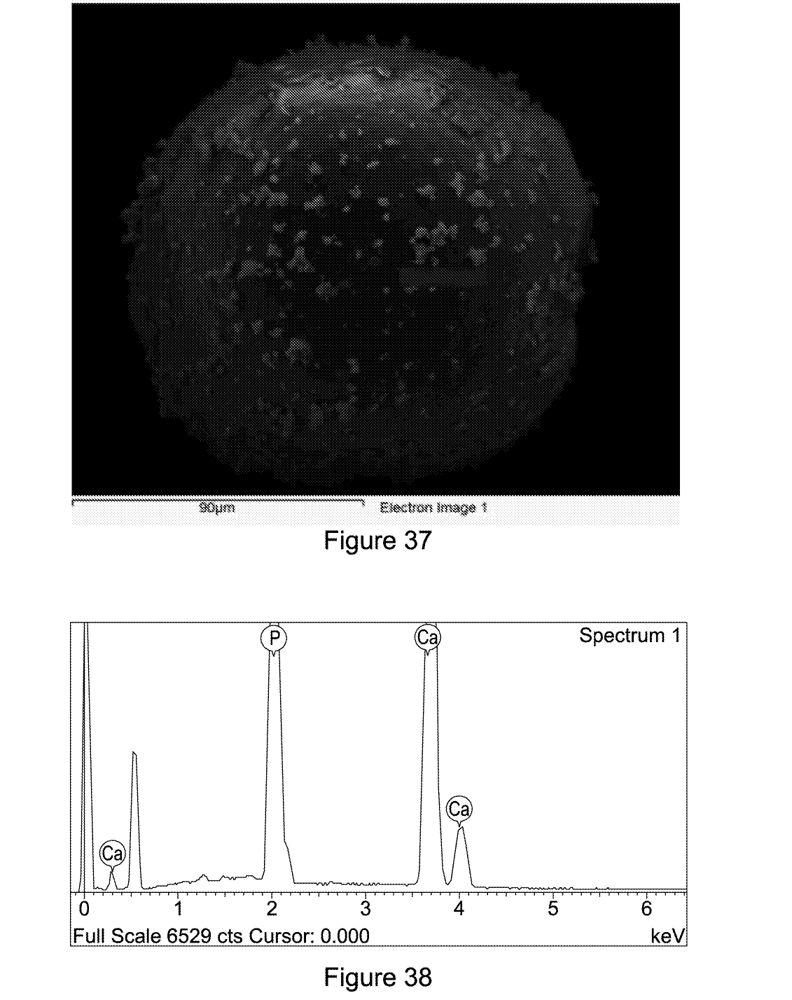

FIG. 37 is an SEM image of a non-porous β-TCP microsphere according to another example embodiment of the invention;

FIG. 38 is an EDX spectrum for the non-porous β-TCP microsphere shown in FIG. 37;

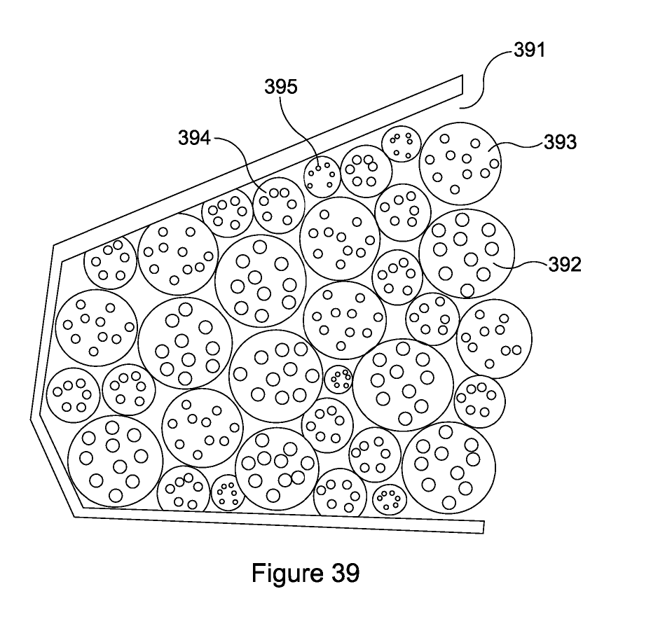

FIG. 39 shows a space filled with porous resorbable microspheres according to the invention;

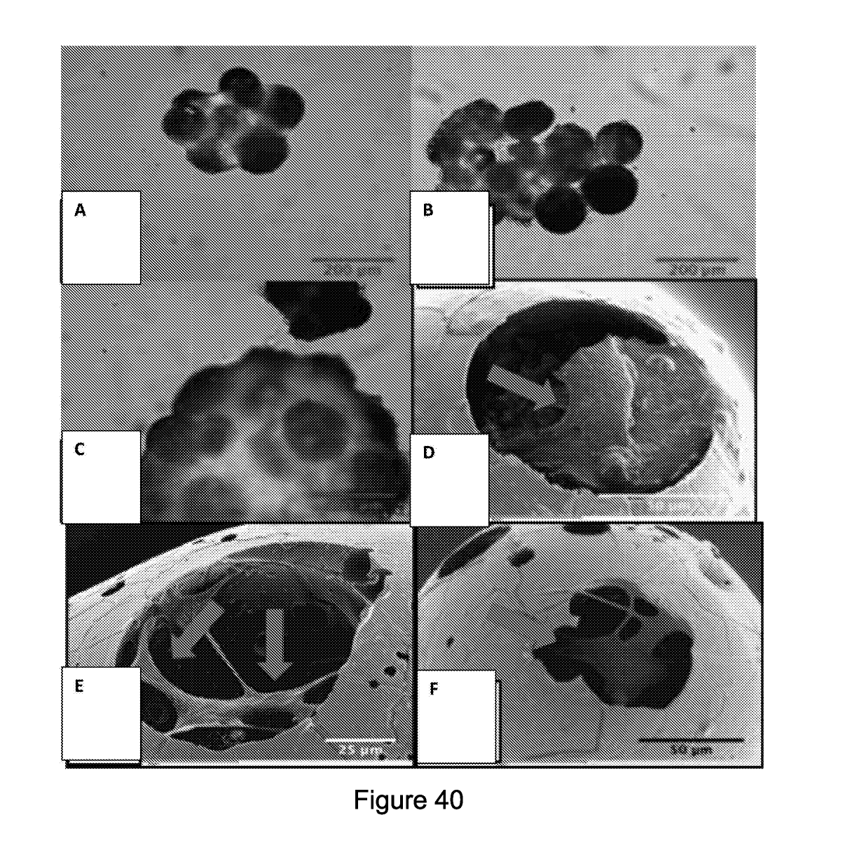

FIG. 40 shows six images of porous calcium phosphate microspheres according to the invention loaded with human mesenchymal stem cells;

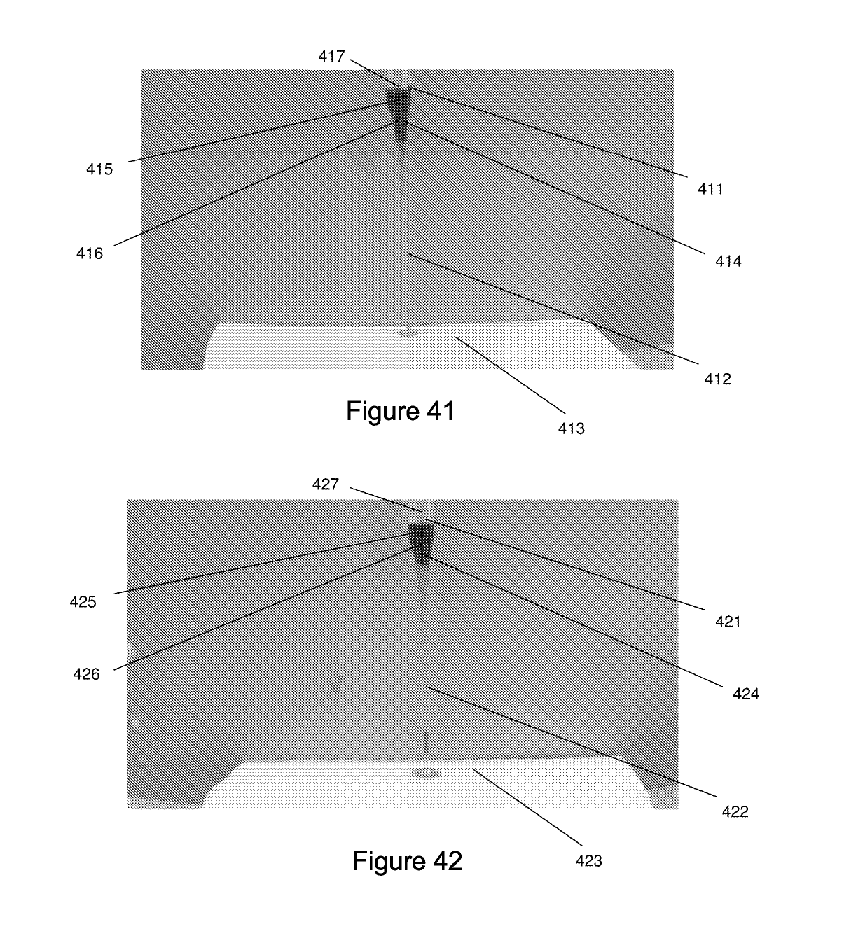

FIG. 41 shows an experiment in which a dye/water solution is passed over irregularly-shaped glass micro particles;

FIG. 42 shows an experiment in which a dye/water solution is passed over bulk (i.e. non-porous) microspheres according to the invention;

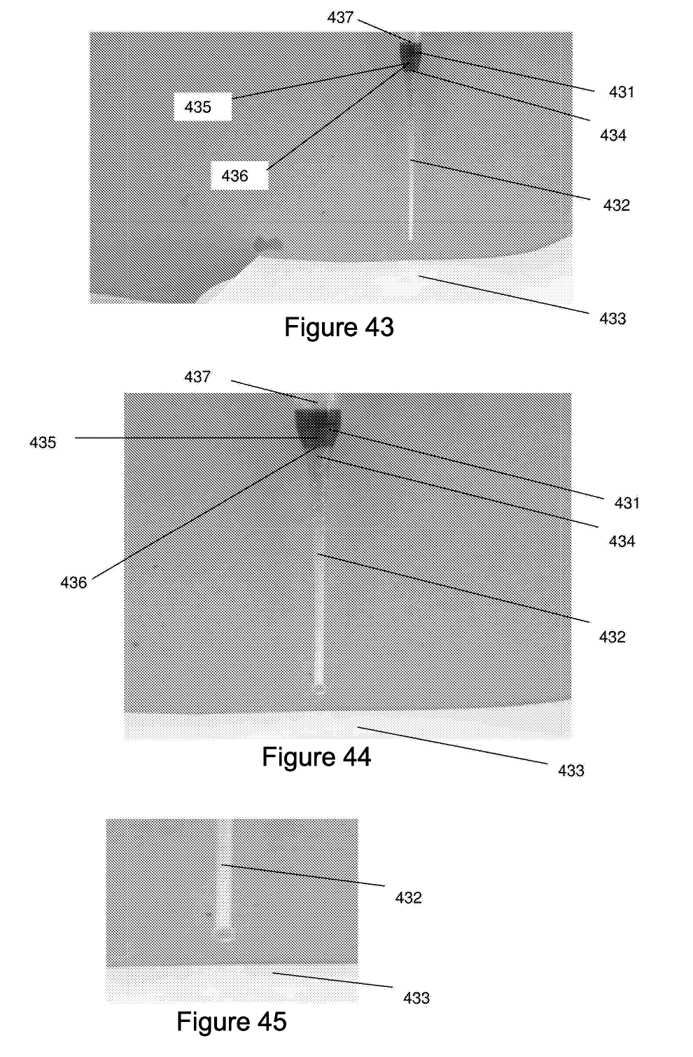

FIGS. 43, 44 and 45 show an experiment in which a dye/water solution is passed over porous microspheres according to the invention; and

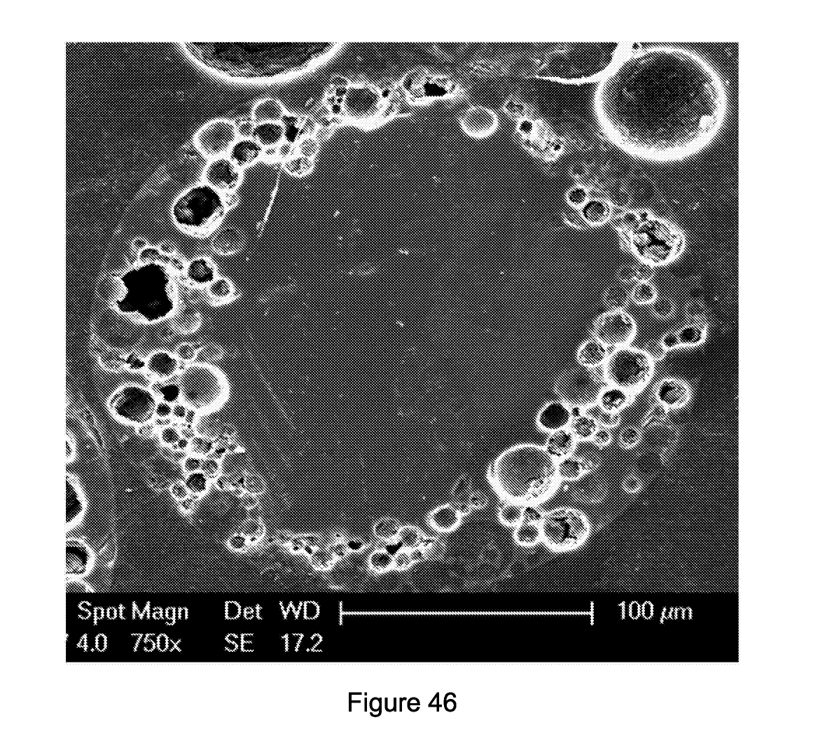

FIG. 46 shows a cross-section of a microsphere according to another example embodiment of the invention, the microsphere having surface porosity and a solid core.

The resorbable porous microspheres shown in FIG. 1, FIG. 2, FIG. 3 and FIG. 4 were manufactured by flame-spraying spheroidisation. A feed powder comprising particles of calcium phosphate glass with a particle size of around 100 μm (±40 μm) was soaked in a solution containing blowing agent(s) (e.g. calcium carbonate and/or sodium sulphate). After soaking, the mixture of the feed powder and the blowing agent(s) was supplied to a spray head and sprayed through a spheroidisation flame produced by an acetylene torch to produce the resorbable porous microspheres of the type shown in FIGS. 1, 2, 3 and 4. Carbon dioxide and/or sulphur dioxide generated from the blowing agent(s) create porosity within the calcium phosphate glass particles.

The resorbable porous microspheres shown in FIG. 1 have a particle size of approximately 140 μm (±50 μm). The yield of resorbable porous microspheres manufactured as described above was in excess of 95%. As can be seen from FIG. 1, the resorbable porous microspheres have very good uniformity.

FIGS. 2, 3 and 4 are further images of the resorbable porous microspheres of FIG. 1. Some areas of interconnected porosity can be seen in FIG. 3.

The porosity of the resorbable porous microspheres of the type shown in FIGS. 1, 2, 3 and 4 was characterised using various techniques including mercury infusion porosimetry. A summary of the results is given in Table 1 below.

TABLE 1

| Average | | Apparent | | Closed | |
|---|---|---|---|---|---|
| pore | Bulk | (skeletal) | Open | porosity | Total |
| diameter | density | density | porosity | (vol %) | porosity |
| (μm) | (g/mL) | (g/mL) | (vol %) | (estimated) | (vol %) |
| 55 | 0.54 | 1.85 | 71 | 9 | 80 |

The resorbable porous microspheres shown in FIG. 5, FIG. 6 and FIG. 7 were manufactured by flame-spraying spheroidisation. A feed powder comprising particles of calcium phosphate glass with a particle size of around 100 μm (±40 μm) was soaked in a solution containing blowing agent(s) (e.g. calcium carbonate and/or sodium sulphate). After soaking, the mixture of the feed powder and the blowing agent(s) was supplied to a spray head and sprayed through a spheroidisation flame produced by an acetylene torch to produce the resorbable porous microspheres of the type shown in FIGS. 5, 6 and 7. Carbon dioxide and/or sulphur dioxide generated from the blowing agent(s) create porosity within the calcium phosphate glass particles.

The resorbable porous microspheres shown in FIG. 5, FIG. 6 and FIG. 7 have a particle size of approximately 140 μm (±50 μm). The yield of resorbable porous microspheres manufactured as described above was in excess of 95%. As can be seen from FIG. 5 and FIG. 6, the resorbable porous microspheres have very good uniformity.

FIGS. 6 and 7 are further images of the resorbable porous microspheres of FIG. 5.

The porosity of the resorbable porous microspheres of the type shown in FIGS. 5, 6 and 7 was characterised using various techniques including mercury infusion porosimetry. A summary of the results is given in Table 2 below.

TABLE 2

| Average | | Apparent | | Closed | |
|---|---|---|---|---|---|
| pore | Bulk | (skeletal) | Open | porosity | Total |
| diameter | density | density | porosity | (vol %) | porosity |
| (μm) | (g/mL) | (g/mL) | (vol %) | (estimated) | (vol %) |
| 56 | 0.58 | 1.66 | 65 | 14 | 79 |

The resorbable porous microspheres shown in FIG. 8, FIG. 9 and FIG. 10 were manufactured by flame-spraying spheroidisation. A feed powder comprising particles of calcium phosphate glass was soaked in a solution containing blowing agent(s) (e.g. calcium carbonate and/or sodium sulphate). After soaking, the mixture of the feed powder and the blowing agent(s) was supplied to a spray head and sprayed through a spheroidisation flame produced by an acetylene torch to produce the resorbable porous microspheres of the type shown in FIGS. 8, 9 and 10. Carbon dioxide and/or sulphur dioxide generated from the blowing agent(s) create porosity within the calcium phosphate glass particles.

The resorbable porous microspheres shown in FIG. 8, FIG. 9 and FIG. 10 have an average particle size of approximately 300 μm. As can be seen from FIGS. 8, 9 and 10, the larger pores typically have a diameter of from 30 μm to 40 μm.

The yield of resorbable porous microspheres manufactured as described above was in excess of 95%. As can be seen from FIG. 8 and FIG. 9, the resorbable porous microspheres have very good uniformity.

FIGS. 9 and 10 are further images of the resorbable porous microspheres of FIG. 8.

The resorbable porous microspheres shown in FIG. 11, FIG. 12 and FIG. 13 were manufactured by flame-spraying spheroidisation. A feed powder comprising particles of calcium phosphate glass was soaked in a solution containing blowing agent(s) (e.g. calcium carbonate and/or sodium sulphate). After soaking, the mixture of the feed powder and the blowing agent(s) was supplied to a spray head and sprayed through a spheroidisation flame produced by an acetylene torch to produce the resorbable porous microspheres of the type shown in FIGS. 11, 12 and 13. Carbon dioxide and/or sulphur dioxide generated from the blowing agent(s) create porosity within the calcium phosphate glass particles.

The resorbable porous microspheres shown in FIG. 11, FIG. 12 and FIG. 13 have an average particle size of approximately 300 μm. As can be seen from FIGS. 11, 12 and 13, the larger pores typically have a diameter of from 30 μm to 40 μm.

The yield of resorbable porous microspheres manufactured as described above was in excess of 95%. As can be seen from FIG. 11, the resorbable porous microspheres have very good uniformity.

FIGS. 12 and 13 are further images of the resorbable porous microspheres of FIG. 11.

Remnants of the blowing agent(s) used in the manufacture of porous resorbable microspheres according to the invention may be incorporated in the microspheres themselves. For instance, energy dispersive x-ray (EDX) analysis of a sample of porous resorbable microspheres according to the invention detected strontium within the microsphere composition, the strontium having come from the blowing agent, strontium carbonate, used in the manufacture of the microspheres. In some embodiments, the blowing agent(s) may be selected, in order to vary and/or finely control doping of the microsphere composition.

Advantageously, the methods of manufacture of the present invention may provide improved yields and/or uniformity of porous resorbable microspheres.

FIG. 14 is a bar chart showing the results of a Brunauer, Emmett and Teller (BET) analysis of the specific surface area (surface area per unit mass) of two example embodiments of porous resorbable microspheres according to the invention compared with the particles of the bulk, substantially non-porous calcium phosphate glass feed powder used in the manufacture of the porous resorbable microspheres.

As indicated by a first column 141, the specific surface area of the bulk, substantially non-porous calcium phosphate glass feed powder was found to be around 0.01 m²/g. As indicated by a second column 142, the specific surface area of porous resorbable microspheres of the type shown in FIGS. 1, 2, 3 and 4 and discussed above was found to be around 0.16 m²/g. As indicated by a third column 143, the specific surface area of porous resorbable microspheres of the type shown in FIGS. 5, 6 and 7 and discussed above was found to be around 0.15 m²/g. The increase in specific surface area that occurs during manufacture of the porous resorbable microspheres from the bulk feed powder (column 141) is around 1200% for the porous resorbable microspheres of column 142 (i.e. porous resorbable microspheres of the type shown in FIGS. 1, 2, 3 and 4 and discussed above) and around 1110% for the porous resorbable microspheres of column 143 (i.e. porous resorbable microspheres of the type shown in FIGS. 5, 6 and 7 and discussed above)

FIG. 15 is an SEM image of a cross-section through a porous resorbable microsphere 151 according to the invention. The porous structure of the porous resorbable microsphere 151 can be seen clearly in FIG. 15. The porous resorbable microsphere 151 contains closed pores, interconnected pores and open, surface pores. An example of a closed pore is labelled 152, an example of an interconnected pore is labelled 153 and an example of an open, surface pore is labelled 154.

FIG. 16 shows a plurality of acid-washed porous calcium phosphate glass microspheres according to another example embodiment of the invention. The microspheres have a diameter of around 100 μm. After the spheroidisation flame had been applied, the calcium phosphate glass microspheres were washed using an acidic solution comprising acetic acid. As can be seen from FIG. 16, the microspheres have relatively larger surface pores and also have a relatively high amount of interconnected porosity.

The data presented in Table 3 below illustrate the effect of washing the microspheres in an acetic acid solution. Two types of microspheres according to the invention were washed in an acetic acid solution. The first type of microspheres (A) were calcium phosphate glass microspheres, having a diameter of approximately 100 μm. The second type of microspheres (B) were calcium phosphate glass microspheres, having a diameter of approximately 100 μm. During manufacture, the ratio by weight of the blowing agent(s) to the calcium phosphate glass particles was different for the two types of microspheres (A and B).

The open porosity of the microspheres was measured pre- and post-wash. The closed porosity of the microspheres was measured pre- and post-wash. Hence, the total porosity of the microspheres could be calculated pre- and post-wash.

For the first type of microspheres (A), washing led to a slight increase in total porosity. Slight increases in the open porosity and/or the closed porosity contributed to the slight increase in total porosity.

For the second type of microspheres (B), washing resulted in a slightly larger increase in total porosity than for the first type of microspheres (A). The increase in total porosity of the second type of microspheres (B) arose, due to a large increase in open porosity, which was offset to some extent by a decrease in closed porosity.

TABLE 3

| | Open porosity | | Closed porosity | | Total porosity | |
|---|---|---|---|---|---|---|
| | (vol %) | | (vol %) | | (vol %) | |
| Micro- | Pre-acid | Post-acid | Pre-acid | Post-acid | Pre-acid | Post-acid |
| spheres | wash | wash | wash | wash | wash | wash |
| A | 71 | 72 (±2) | 9 | 10 (±3) | 80 | 82 (±1) |
| B | 65 | 76 (±2) | 14 | 7 (±3) | 79 | 83 (±2) |

Without wishing to be bound by any theory, it is thought that washing the microspheres in acetic acid solution removes residual blowing agent(s) from the microspheres. The removal of residual blowing agent(s) may contribute at least partially to an increase in total porosity of the microspheres. Pores that were closed or obstructed due to the presence of residual blowing agent(s) may be opened as a result of the washing.

FIG. 17 is an SEM image of a plurality of non-porous borosilicate microspheres according to another example embodiment of the invention. The non-porous borosilicate microspheres were manufactured by flame spraying spheroidisation using an acetylene torch. The non-porous borosilicate microspheres have a diameter of approximately 85 μm and have very good sphericity.

FIG. 18 is an EDX spectrum for the non-porous borosilicate microspheres shown in FIG. 17.

FIG. 19 is an SEM image of a porous borosilicate microsphere according to another example embodiment of the invention. The porous borosilicate microspheres were manufactured by flame spraying spheroidisation using an acetylene torch. The porous borosilicate microspheres have good sphericity.

FIG. 20 is an EDX spectrum for the porous borosilicate microspheres shown in FIG. 19.

FIG. 21 is an SEM image of a plurality of non-porous Bioglass® microspheres according to another example embodiment of the invention. The non-porous Bioglass® microspheres were manufactured by flame spraying spheroidisation using an acetylene torch. The non-porous Bioglass® microspheres have very good sphericity. The non-porous Bioglass® microspheres have a range of diameters, from approximately 40 μm to approximately 200 μm. A significant number of the non-porous Bioglass® microspheres shown in FIG. 21 have a diameter of approximately 110 μm, while another significant number of the Bioglass® microspheres shown in FIG. 21 have a diameter of approximately 120 μm.

FIG. 22 is an EDX spectrum for the non-porous Bioglass® microspheres shown in FIG. 21.

FIG. 23 is an SEM image of a plurality of porous Bioglass® microspheres according to another example embodiment of the invention. The porous Bioglass® microspheres were manufactured by flame spraying spheroidisation using an acetylene torch. The porous Bioglass® microspheres have good sphericity.

FIG. 24 is an EDX spectrum for the porous Bioglass® microspheres shown in FIG. 23. The Zr in the EDX spectrum may be from contamination during grinding in a ball mill. The appearance of Sr in the EDX spectrum was surprising and unexpected; it could be due to overlap in Ca/Sr peaks or contamination.

FIG. 25 is an SEM image of a plurality of non-porous borate glass microspheres according to another example embodiment of the invention. The non-porous borate glass microspheres were manufactured by flame spraying spheroidisation using an acetylene torch. The non-porous borate glass microspheres have very good sphericity. The non-porous borate glass microspheres have diameters of from approximately 150 μm to approximately 250 μm.

FIG. 26 is an EDX spectrum for the non-porous borate glass microspheres shown in FIG. 25.

FIG. 27 is an SEM image of a plurality of porous borate glass microspheres according to another example embodiment of the invention. The porous borate glass microspheres were manufactured by flame spraying spheroidisation using an acetylene torch. The porous borate glass microspheres have good sphericity. The porous borate glass microspheres have diameters of from approximately 220 μm to approximately 250 μm.

FIG. 28 is an EDX spectrum for the non-porous borate glass microspheres shown in FIG. 27.

FIG. 29 is an SEM image of a plurality of non-porous apatite wollastonite microspheres according to another example embodiment of the invention. The non-porous apatite wollastonite microspheres were manufactured by flame spraying spheroidisation using an acetylene torch. The non-porous apatite wollastonite microspheres have good sphericity. The non-porous apatite wollastonite microspheres have a range of diameters, from approximately 100 μm to approximately 140 μm.

FIG. 30 is an EDX spectrum for the non-porous apatite wollastonite microspheres shown in FIG. 29.

FIG. 31 is an SEM image of a plurality of porous apatite wollastonite microspheres according to another example embodiment of the invention. The porous apatite wollastonite microspheres were manufactured by flame spraying spheroidisation using an acetylene torch. The porous apatite wollastonite microspheres have good sphericity. The porous apatite wollastonite microspheres have a range of diameters, from approximately 100 μm to approximately 170 μm.

FIG. 32 is an EDX spectrum for the non-porous apatite wollastonite microspheres shown in FIG. 31.

FIG. 33 is an SEM image of a plurality of non-porous hydroxyapatite microspheres according to another example embodiment of the invention. The non-porous hydroxyapatite microspheres were manufactured by flame spraying spheroidisation using an acetylene torch. The non-porous hydroxyapatite microspheres have good sphericity. The non-porous hydroxyapatite microspheres have a range of diameters, from approximately 100 μm to approximately 160 μm.

FIG. 34 is an EDX spectrum for the non-porous hydroxyapatite microspheres shown in FIG. 33.

FIG. 35 is an SEM image of a hollow hydroxyapatite microsphere according to another example embodiment of the invention. The hollow hydroxyapatite microsphere was manufactured by flame spraying spheroidisation using an acetylene torch. The hollow hydroxyapatite microsphere has good sphericity. The hollow hydroxyapatite microsphere has a diameter of approximately 120 μm.

FIG. 36 is an EDX spectrum for the hollow hydroxyapatite microsphere shown in FIG. 35.

FIG. 37 is an SEM image of a non-porous β-TCP microsphere according to another example embodiment of the invention. The non-porous β-TCP microsphere was manufactured by flame spraying spheroidisation using an acetylene torch. The non-porous β-TCP microsphere has good sphericity. The non-porous β-TCP microsphere has a diameter of approximately 150 μm.

FIG. 38 is an EDX spectrum for the non-porous β-TCP microspheres shown in FIG. 37.

A better packing efficiency may be achieved by providing microspheres of a plurality of different sizes.

FIG. 39 shows an irregularly-shaped space 391 filled with a powder comprising resorbable porous microspheres according to the invention. The powder comprises four different, distinctly-sized, types of resorbable porous microspheres according to the invention. A first type of resorbable porous microsphere 392 has a larger diameter than a second type of resorbable porous microsphere 393, which has a larger diameter than a third type of resorbable porous microsphere 394, which has a larger diameter than a fourth type of resorbable porous microsphere 395.

A powder comprising resorbable porous microspheres according to the invention may contain any number of types of porous resorbable microspheres mixed in any ratio. Accordingly, a powder may be produced having particles of more than one distinct particle size distribution and/or porosity.

The powder may have any particle size distribution. For instance, the powder may have a monomodal, bimodal, trimodal, tetramodal, pentamodal or hexamodal particle size distribution. Different particle size distributions may be better suited for different applications.

The irregularly-shaped space could be, for example, a space between two sections of bone or a defect or void within a bone.

One application for resorbable porous microspheres of the invention is in bone tissue regeneration, e.g. in the treatment of osteoporosis or other bone resorption disorders.

For this application, calcium phosphate microspheres according to the invention may be loaded with autologous stem cells (or other cell types) and/or other biological components. The resorbable porous microspheres of the invention could be used as a bone graft substitute.

Osteoporosis and fragility fractures are a major problem worldwide, particularly in countries with aging populations. As a consequence, there is an ever-growing need for long-term orthopaedic care.

The present invention may help to facilitate a shift from tissue repair to tissue regeneration. By facilitating a shift from tissue repair to tissue regeneration, the growth rate of the need for long-term orthopaedic care may be reduced.

Across Europe, an estimated four million new fractures occur per year (around eight fractures each minute or one every eight seconds). The total direct cost of these fractures has been estimated at [symbol] 31.7 billion, which is forecast to increase to [symbol] 76.7 billion by 2050 based on anticipated changes in the demography of Europe.

In the UK, the annual combined healthcare and social cost for fractures in bones weakened by osteoporosis is nearly £1.73 billion.

In the UK, currently nearly 20 million people are aged 50 or more. This is predicted to increase to 25 million by 2020. Over 60,000 hip, 50,000 wrist and 120,000 vertebral osteoporosis-related fractures occur each year in the UK. According to the National Osteoporosis Society, recent trends suggest that hip fracture rates will increase to 117,000 by 2016.

In 2001, combined NHS and social care costs for a single hip fracture in the UK were estimated to be £20000.

Each year fractures in patients aged 60 and over account for more than two million hospital bed days in England alone. Around 30% of over 65 year olds living in the community will fall each year (increasing to 42% for the over 75 age group), while over 60% of people in care homes fall each year.

Usually, treatment is not administered until after a person, e.g. an elderly person with osteoporosis, has suffered a broken bone. Advantageously, treatment using the present invention may be administered prior to any fractures (or any further fractures) occurring, in order to reduce the likelihood of an individual suffering a fracture in a bone weakened by osteoporosis. Apart from patient benefits, this may also lead to significant social and healthcare cost savings.

In an example embodiment of the invention, an individual may be identified as having or being at risk of having osteoporosis. For instance, the individual may have suffered (or be at risk of suffering) a fracture, e.g. an osteoporotic compression fracture. The individual may then have an examination, typically an x-ray examination, in order to identify any regions of resorbed osteoporotic bone. The examination, e.g. the x-ray examination, may comprise a whole-body scan. A whole-body scan may be able to provide information on overall and local bone mineral content (BMC) and bone mineral density (BMD).

Autologous stem cells may then be isolated from the individual. The autologous stem cells isolated from the individual are then loaded into bio-resorbable porous microspheres according the invention.

The bio-resorbable porous microspheres loaded with the autologous stem cells may then be injected into the identified region(s) of resorbed osteoporotic bone. Typically, this may involve only a minimally invasive surgical procedure using needles or cannulae. Accordingly, the individual may be treated as a hospital day-case patient.

The bio-resorbable porous microspheres may dissolve over time within the body, without causing any harm to the individual. The autologous stem cells will act to promote regeneration of bone tissue, thereby strengthening the identified region(s) of resorbed osteoporotic bone. Hence, the likelihood of the individual suffering a bone fracture may be reduced.

An individual may be found to have a region of resorbed osteoporotic bone. The region of resorbed osteoporotic bone could be in any part of the individual's skeleton, e.g. the spine, femur, hip, ankle or wrist. A syringe or cannula may be used to inject porous bio-resorbable microspheres loaded with autologous stem cells isolated from the individual into the region of resorbed osteoporotic bone.

It will be appreciated that the porous resorbable microspheres of the invention may provide an osteoporotic fracture prevention prophylactic. Advantageously, this preventative treatment may be delivered non-invasively or via a minimally invasive surgical procedure.

While dissolving within the body, the bio-resorbable porous microspheres may release active and/or therapeutic agents, e.g. ions, other than, or as well as, cells such as autologous stem cells.

The applicant has carried out experiments in which human mesenchymal stem cells (hMSC) have been loaded into porous calcium phosphate glass microspheres according to the invention. Porous resorbable microspheres comprising pores having larger diameters may be preferred for applications in which the porous resorbable microspheres are loaded with stem cells.

FIG. 40 includes six images (labelled A, B, C, D, E and F) of calcium phosphate microspheres loaded with human mesenchymal stem cells (hMSC). Images A, B and C show an in vitro multicellular hMSC aggregate formation incorporating porous calcium phosphate microspheres. Images D, E and F are SEM images of human mesenchymal stem cells within the pores of the calcium phosphate microspheres. The arrows in images D, E and F point towards the human mesenchymal stem cells.

Porous microspheres according to the invention may be loaded with agents other than stem cells, e.g. cells, growth factors, proteins or pharmaceutically active agents.

The porous microspheres of the invention may have utility in the treatment of fractures, e.g. osteoporotic fractures such as osteoporotic vertebral fractures.

The porous microspheres of the invention may find utility as a bone graft material.

Application of the porous resorbable microspheres of the invention is not limited to bone regeneration.

The porous resorbable microspheres may be loaded with chemical or biological drugs or other active agents for release into the human or animal body.

The invention may also have utility in non-biomedical applications. An example of a non-biomedical application is filtration and separation.

For instance, porous microspheres may be used to separate a mixture of large and small molecules. An initial mixture of larger molecules and smaller molecules is fed to a gel filtration resin comprising a plurality of porous microspheres according to the invention. The smaller molecules may be “included” (i.e. be small enough to pass into and through the pores of porous microspheres), while the larger molecules may be “excluded” (i.e. be too large to enter the pores of the porous microspheres 203). Hence, the larger molecules 201 may be eluted before the smaller molecules 202.

Another potential use for porous resorbable microspheres according to the invention is as a replacement for “microbeads” which are found in many beauty and cleaning products.

Microbeads are typically made from plastic and are included in products such as shower gel, face washes, toothpaste and cleaning products for their abrasive qualities.

A problem with microbeads is that typically they may be too small to be filtered out at water treatment plants and consequently may end up in lakes and rivers. The plastic may soak up toxins and be eaten by fish and other creatures. In this way, there is a concern that toxins may build up in the food chain and eventually be consumed by humans.

Porous resorbable microspheres according to the present invention may be used as a substitute for microbeads. They could provide the required abrasive qualities before dissolving harmlessly into the environment, e.g. in a lake or river, in a fish or other creature or in a human or animal at the top of the food chain.

Other potential applications for microspheres according to the invention may include: use as a feedstock for additive manufacturing; filtration; separation; fluid, e.g. water, purification; beauty and personal care products such as cosmetics, shower gel and face wash; laundry and cleaning products; or use in applications requiring a lightweight low thermal expansion and low conductivity material.

For example, porous and/or non-porous microspheres, e.g. glass or glass-ceramic bulk (i.e. non-porous) and/or porous microspheres according to the invention may be used in combination with other particles or bodies such as microspheres. Such other particles or bodies may comprise polymer microspheres.

In an example embodiment, porous and/or non-porous microspheres according to the invention may be used in combination with natural or synthetic polymer microspheres. The utilisation of natural or synthetic polymer microspheres may help to achieve control over drug and/or biological component release. Additionally or alternatively, the natural or synthetic polymer microspheres may be utilised to deliver alternate drugs or biological components directly to sites of interest.

In another example embodiment, combining fast-resorbing microspheres, e.g. glass microspheres such as calcium phosphate microspheres, according to the invention with polymer microspheres could be used to achieve control over the polymer degradation profiles and vice versa—acidic release from polymer microspheres could be used to control release from the glass microspheres.

In another example embodiment, microspheres according to the invention, e.g. bulk (i.e. non-porous) and/or porous glass or glass-ceramic microspheres, may be coated at least partially with one or more resorbable (natural or synthetic) polymers to gain improved control over release of components carried by, e.g. encapsulated within and/or coated on, the microspheres.

Gaining control of particle geometry can be critical for additive manufacturing (e.g. 3D printing). Accordingly, microspheres according to the invention may be well suited for use in a feedstock for an additive manufacturing process, due to their uniformity and/or sphericity.

Currently, for instance, there is significant interest in possible additive manufacturing of biological materials or components, but it is proving extremely difficult to achieve and/or optimise. In an example embodiment, porous microspheres according to the invention may be loaded with one or more biological components (or non-biological entities) of interest to provide a feedstock. The feedstock may then be supplied to a 3D printer or other additive manufacturing device operable to produce a component having a desired geometry. With careful control over the composition of the microspheres, e.g. glass formulations of the microspheres, the component could then be engineered to degrade away (over time and/or in situ), leaving behind the incorporated biological components.

Microspheres according to the present invention may be utilised in separation and/or filtration applications.

In an example, it is envisaged that microspheres according to the invention may have utility in filtration devices within industrial applications (such as desalination plants), e.g. to remove heavy metals or bacteria.

FIG. 41 shows an experiment in which a dye/water solution 415 is passed over ground irregularly-shaped, non-porous glass micro-particles 414. A vertically-oriented glass micropipette 417 having an upper, wider portion 411 and a lower, thinner portion 412 is arranged above a blotting surface 413. A tapered portion 416 connects the upper, wider portion 411 to the lower, thinner portion 412. The irregularly-shaped glass micro-particles 414 are provided within the tapered portion 416. In the experiment, the dye/water solution 415 is passed through the micropipette 417. It took five minutes for the solution to pass through the micropipette 417 on to the blotting surface 413.

FIG. 42 shows an experiment in which a dye/water solution 425 is passed over non-porous microspheres according to the invention 424. A vertically-oriented glass micropipette 427 having an upper, wider portion 421 and a lower, thinner portion 422 is arranged above a blotting surface 423. A tapered portion 426 connects the upper, wider portion 421 to the lower, thinner portion 422. The non-porous microspheres according to the invention 424 are provided within the tapered portion 426. In the experiment, the dye/water solution 425 is passed through the micropipette 427. It took around 90 seconds for the solution to pass through the micropipette 427 on to the blotting surface 423.

The solution passed through the micropipette much quicker in the experiment shown in FIG. 42 than in the experiment shown in FIG. 41. Without wishing to be bound by any theory, it is postulated that the uniformity and sphericity of the non-porous microspheres according to the invention 424, as compared with the irregularly-shaped glass micro-particles 414, was responsible in large part for the significant increase in the flow rate of the dye/water solution through the micropipette.

It is noted that in both FIG. 41 and FIG. 42, the dye/water solution is not separated as it passes through the micropipette.

FIGS. 43, 44 and 45 show an experiment in which a dye/water solution 435 is passed over porous microspheres according to the invention 434. FIGS. 44 and 45 are magnified views of portions of FIG. 43. A vertically-oriented glass micropipette 437 having an upper, wider portion 431 and a lower, thinner portion 432 is arranged above a blotting surface 433. A tapered portion 436 connects the upper, wider portion 431 to the lower, thinner portion 432. The porous microspheres according to the invention 434 are provided within the tapered portion 436. In the experiment, the dye/water solution 435 is passed through the micropipette 437.

In contrast with the experiments shown in FIGS. 41 and 42, the dye/water solution 435 is separated as it passes through the porous microspheres according to the invention 434. As can be seen in FIGS. 43, 44 and 45, the dye is retained in the upper, wider portion 431 of the micropipette 437 and only water exits the bottom of the micropipette 437 on to the blotting surface 435.

Accordingly, the porous microspheres according to the invention are capable of separating and retaining the dye from the water. This result suggests that with careful control over the chemistry and/or composition of the porous microspheres, e.g. porous glass or glass-ceramic microspheres, the internal surfaces of the porous microspheres could be adapted to filter out specific entities, e.g. heavy metals or other unwanted entities in solutions.

FIG. 46 shows a polished cross-section of a calcium phosphate glass microsphere according to the invention. The calcium phosphate glass microsphere has a solid core and surface porosity.

1. A method of manufacture of a powder comprising microspheres, the method comprising:
providing a feed powder; and
applying at least one spheroidisation flame to the powder.

2. The method according to claim 1 comprising the steps of:
mixing the feed powder with one or more blowing agents to provide a mixture; and
applying at least one spheroidisation flame to the mixture.

3. The method according to claim 1, wherein the feed powder comprises porous and/or non-porous particles.

4. The method according to claim 1, wherein the feed powder comprises substantially spherical and/or non-spherical particles.

5. The method according to claim 1, wherein the feed powder has an average particle size of from 30 μm to 500 μm.

6. The method according to claim 1 comprising flame-spraying spheroidisation.

7. The method according to claim 1, wherein the spheroidisation flame temperature is from 1900° C. to 3400° C.

8. The method according to claim 2, wherein the blowing agent has an average particle size of at least 5 μm and/or up to 500 μm.

9. The method according to claim 2, wherein the blowing agent comprises a carbonate or a sulphate.

10. The method according to claim 2, wherein the mixture is produced prior to applying the spheroidisation flame or at the same time as applying the spheroidisation flame.

11. The method according to claim 2, wherein the method comprises the step of soaking the feed powder in a solution containing the blowing agent(s).

12. The method according to claim 2, wherein the method comprises a washing step to remove residual blowing agent.

13. A method of manufacture of a powder comprising microspheres, the method comprising:
manufacturing a first powder according to the method of claim 1;
manufacturing at least one further powder according to the method of claim 1, wherein the at least one further powder contains particles having a different size distribution and/or a different porosity from the first powder; and
mixing the first powder and the at least one further powder together.

14. A powder according to claim 1 comprising porous and/or non-porous microspheres.

15. The powder according to claim 14, wherein the microspheres have an average particle size of at least 30 μm and/or up to 500 μm.

16. The powder according to claim 14, wherein the microspheres comprise a resorbable composition or a non-resorbable composition.

17. The powder according to claim 14, wherein the microspheres comprise a glass, a glass-ceramic or a ceramic composition.

18. The powder according of claim 14, wherein the microspheres comprise a phosphate-based glass.

19. The powder according to claim 14, wherein the microspheres have a surface area per unit mass of at least 0.05 m²/g.

20. The powder according to claim 14, wherein the microspheres are porous and the average pore diameter is at least 10 μm and/or up to 100 μm.

21. The powder according to claim 14, wherein the microspheres have a total porosity of at least 40%.

22. The powder according to claim 14, wherein the powder comprises a mixture of a first powder and at least one further powder, and wherein the first powder comprises microspheres having a first size distribution and the further powder comprises microspheres having a different size distribution.

23. The powder according to claim 22, wherein the microspheres of the first powder are smaller than the microspheres of a second powder.

24. The powder according to claim 23, wherein the microspheres of the first powder have an average particle size of up to 200 μm and/or the microspheres of the second powder have an average particle size of more than 200 μm.

25. The powder according to claim 14, wherein the microspheres are coated and/or loaded with at least one active agent.

26. The powder according to claim 1, wherein the microspheres are coated and/or impregnated/doped with an anti-microbial agent.

27. A method for delivering an active agent comprising utilizing a powder according to claim 14.

28. The method according to claim 9, wherein the carbonate or the sulphate is a calcium carbonate, a strontium carbonate, or a sodium sulphate.

29. The powder according to claim 18, wherein the phosphate-based glass is selected from the group consisting of a calcium phosphate-based glass, a Bioglass®, hydroxyapatite, a tri-calcium phosphate (α-TCP), a β tri-calcium phosphate (β-TCP), a borosilicate glass, a borate glass, an apatite wollastonite, and a combination thereof.

30. The powder according to claim 19, wherein the microspheres have a surface area per unit mass of up to or at least 0.08 m²/g.

31. The powder according to claim 30, wherein the microspheres have a surface area per unit mass of up to or at least 0.12 m²/g.

32. The powder according to claim 31, wherein the microspheres have a surface area per unit mass of up to or at least 0.14 m²/g.

33. The powder according to claim 21, wherein the microspheres have a total porosity of at least 50%.

34. The powder according to claim 33, wherein the microspheres have a total porosity of at least 60%.

35. The powder according to claim 34, wherein the microspheres have a total porosity of at least 70%.

36. The powder according to claim 35, wherein the microspheres have a total porosity of at least 80%.

37. The powder according to claim 25, wherein the at least one active agent is selected from the list consisting of a pharmaceutically active agent, a biological cell, a growth factor, a protein, and a combination thereof.

38. The powder according to claim 37, wherein the biological cell is a stem cell.

39. The powder according to claim 26, wherein the anti-microbial agent is selected from the list consisting of silver, zinc, copper, and a combination thereof.
